SEQUENCE LISTING (3 sequences)

SEQ ID NO: 1 - DNA, 2947 bp, Gallus gallus
aaaccaatatatttccaaatgaaaaaaaaatctgataaaaagttgactttaaaaaaggtatcaataaatgtatgcatttctcactagccttaaactctgcatgaagtgtttgatgagcagatgaagacaacatcatttctagtttcagaaataataacagcatcaaaaccgcagctgtaactccactgagctcacgttaagttttgatgtgtgaatatctgacagaactgacataatgagcactgcaaggatatcagacaagtcaaaatgaagacagacaaaagtattttttaatataaaaatggtctttatttcttcaatacaaggtaaactactattgcagtttaagaccaacacaaaagttggacagcaaattgcttaacagtctcctaaaggctgaaaaaaaggaacccatgaaagctaaaagttatgcagtatttcaagtataacatctaaaaatgatgaaacgatccctaaaggtagagattaactaagtacttctgctgaaaatgtattaaaatccgcagttgctaggataccatcttaccttgttgagaaatacaggtctccggcaacgcaacattcagcagactctttggcctgctggaatcaggaaactgcttactatatacacatataaatcctttggagttgggcattctgagagacatccatttcctgacattttgcagtgcaactctgcattccaactcagacaagctcccatgctgtatttcaaagccatttcttgaatagtttacccagacatccttgtgcaaattgggaatgaggaaatgcaatggtacaggaagacaatacagccttatgtttagaaagtcagcagcgctggtaatcttcataaaaatgtaactgttttccaaataggaatgtatttcacttgtaaaacacctggtcctttttatattacttttttttttttttaaggacacctgcactaatttgcaatcacttgtatttataaaagcacacgcactcctcattttcttacatttgaagatcagcagaatgtctctttcataatgtaataatcatatgcacagtttaaaatattttctattacaaaatacagtacacaagagggtgaggccaaagtctattacttgaatatattccaaagtgtcagcactgggggtgtaaaattacattacatggtatgaataggcggaattcttttacaactgaaatgctcgatttcattgggatcaaaggtaagtactgtttactatcttcaagagacttcaatcaagtcggtgtatttccaaagaagcttaaaagattgaagcacagacacaggccacaccagagcctacacctgctgcaataagtggtgctatagaaaggattcaggaactaacaagtgcataatttacaaatagagatgctttatcatactttgcccaacatgggaaaaaagacatcccatgagaatatccaactgaggaacttctctgtttcatagtaactcatctactactgctaagatggtttgaaaagtacccagcaggtgagatatgttcgggaggtggctgtgtggcagcgtgtcccaacacgacacaaagcaccccacccctatctgcaatgctcactgcaaggcagtgccgtaaacagctgcaacaggcatcacttctgcataaatgctgtgactcgttagcatgctgcaactgtgtttaaaacctatgcactccgttaccaaaataatttaagtcccaaataaatccatgcagcttgcttcctatgccaacatattttagaaagtattcattcttctttaagaatatgcacgtggatctacacttcctgggatctgaagcgatttatacctcagttgcagaagcagtttagtgtcctggatctgggaaggcagcagcaaacgtgcccgttttacatttgaacccatgtgacaacccgccttactgagcatcgctctaggaaatttaaggctgtatccttacaacacaagaaccaacgacagactgcatataaaattctataaataaaaataggagtgaagtctgtttgacctgtacacacagagcatagagataaaaaaaaaaggaaatcaggaattacgtatttctataaatgccatatatttttactagaaacacagatgacaagtatatacaacatgtaaatccgaagttatcaacatgttaactaggaaaacatttacaagcatttgggtatgcaactagatcatcaggtaaaaaatcccattagaaaaatctaagcctcgccagtttcaaaggaaaaaaaccagagaacgctcactacttcaaaggaaaaaaaataaagcatcaagctggcctaaacttaataaggtatctcatgtaacaacagctatccaagctttcaagccacactataaataaaaacctcaagttccgatcaacgttttccataatgcaatcagaaccaaaggcattggcacagaaagcaaaaagggaatgaaagaaaagggctgtacagtttccaaaaggttcttcttttgaagaaatgtttctgacctgtcaaaacatacagtccagtagaaattttactaagaaaaaagaacaccttacttaaaaaaaaaaaacaacaaaaaaaacaggcaaaaaaacctctcctgtcactgagctgccaccacccaaccaccacctgctgtgggctttgtctcccaagacaaaggacacacagccttatccaatattcaacattacttataaaaacgctgatcagaagaaataccaagtatttcctcagagactgttatatcctttcatcggcaacaagagatgaaatacaacagagtgaatatcaaagaaggcggcaggagccaccgtggcaccatcaccgggcagtgcagtgcccaactgccgttttctgagcacgcataggaagccgtcagtcacatgtaataaaccaaaacctggtacagttatattat

SEQ ID NO: 2 - DNA, 11169 bp, Artificial
  A. thaliana plant vector comprising T-DNA region, pFAJ3160
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaggcctgtcgacgcccgggcggtaccgcgatcgctcgcgacctgcaggcataaagccgtcagtgtccgcataaagaaccacccataatacccataatagctgtttgccatcgctaccttaggaccgttatagttaaccggtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggccggccgagctcggtagcaattcccgaggctgtagccgacgatggtgccaccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacataagggactgacctacccggggatcctctagagccatggtgtttaaacgttaactgtaattgtaaatagtaattgtaatgttgtttgttgtttgttgttgttggtaattgttgtaaaaatactcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttcttttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttcttttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttcaagcttgcggccgctcgctaccttaggaccgttatagttaattaccctgttatccctattaattaagagctcgctaccttaagagaggatatcggcgcgccgaattcgcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagctggaagatttgatggatcctcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatctgtttaaacgttaacggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaatcgttgcggttctgtcagttccaaacgtaaaacggcttgtcccgcgtcatcggcgggggtcataacgtgactcccttaattctccgctcatgatcaagcttggcgcgcctctagaatttaaatggatcctacgtactcgagaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 3 - DNA, 17062 bp, Artificial
  A. thaliana plant transformation vector with chicken lysozyme gene flanking the T-DNA region, pFAJ3163
atttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaggcctgtcgactctagaaaaccaatatatttccaaatgaaaaaaaaatctgataaaaagttgactttaaaaaaggtatcaataaatgtatgcatttctcactagccttaaactctgcatgaagtgtttgatgagcagatgaagacaacatcatttctagtttcagaaataataacagcatcaaaaccgcagctgtaactccactgagctcacgttaagttttgatgtgtgaatatctgacagaactgacataatgagcactgcaaggatatcagacaagtcaaaatgaagacagacaaaagtattttttaatataaaaatggtctttatttcttcaatacaaggtaaactactattgcagtttaagaccaacacaaaagttggacagcaaattgcttaacagtctcctaaaggctgaaaaaaaggaacccatgaaagctaaaagttatgcagtatttcaagtataacatctaaaaatgatgaaacgatccctaaaggtagagattaactaagtacttctgctgaaaatgtattaaaatccgcagttgctaggataccatcttaccttgttgagaaatacaggtctccggcaacgcaacattcagcagactctttggcctgctggaatcaggaaactgcttactatatacacatataaatcctttggagttgggcattctgagagacatccatttcctgacattttgcagtgcaactctgcattccaactcagacaagctcccatgctgtatttcaaagccatttcttgaatagtttacccagacatccttgtgcaaattgggaatgaggaaatgcaatggtacaggaagacaatacagccttatgtttagaaagtcagcagcgctggtaatcttcataaaaatgtaactgttttccaaataggaatgtatttcacttgtaaaacacctggtcctttttatattacttttttttttttttaaggacacctgcactaatttgcaatcacttgtatttataaaagcacacgcactcctcattttcttacatttgaagatcagcagaatgtctctttcataatgtaataatcatatgcacagtttaaaatattttctattacaaaatacagtacacaagagggtgaggccaaagtctattacttgaatatattccaaagtgtcagcactgggggtgtaaaattacattacatggtatgaataggcggaattcttttacaactgaaatgctcgatttcattgggatcaaaggtaagtactgtttactatcttcaagagacttcaatcaagtcggtgtatttccaaagaagcttaaaagattgaagcacagacacaggccacaccagagcctacacctgctgcaataagtggtgctatagaaaggattcaggaactaacaagtgcataatttacaaatagagatgctttatcatactttgcccaacatgggaaaaaagacatcccatgagaatatccaactgaggaacttctctgtttcatagtaactcatctactactgctaagatggtttgaaaagtacccagcaggtgagatatgttcgggaggtggctgtgtggcagcgtgtcccaacacgacacaaagcaccccacccctatctgcaatgctcactgcaaggcagtgccgtaaacagctgcaacaggcatcacttctgcataaatgctgtgactcgttagcatgctgcaactgtgtttaaaacctatgcactccgttaccaaaataatttaagtcccaaataaatccatgcagcttgcttcctatgccaacatattttagaaagtattcattcttctttaagaatatgcacgtggatctacacttcctgggatctgaagcgatttatacctcagttgcagaagcagtttagtgtcctggatctgggaaggcagcagcaaacgtgcccgttttacatttgaacccatgtgacaacccgccttactgagcatcgctctaggaaatttaaggctgtatccttacaacacaagaaccaacgacagactgcatataaaattctataaataaaaataggagtgaagtctgtttgacctgtacacacagagcatagagataaaaaaaaaaggaaatcaggaattacgtatttctataaatgccatatatttttactagaaacacagatgacaagtatatacaacatgtaaatccgaagttatcaacatgttaactaggaaaacatttacaagcatttgggtatgcaactagatcatcaggtaaaaaatcccattagaaaaatctaagcctcgccagtttcaaaggaaaaaaaccagagaacgctcactacttcaaaggaaaaaaaataaagcatcaagctggcctaaacttaataaggtatctcatgtaacaacagctatccaagctttcaagccacactataaataaaaacctcaagttccgatcaacgttttccataatgcaatcagaaccaaaggcattggcacagaaagcaaaaagggaatgaaagaaaagggctgtacagtttccaaaaggttcttcttttgaagaaatgtttctgacctgtcaaaacatacagtccagtagaaattttactaagaaaaaagaacaccttacttaaaaaaaaaaaacaacaaaaaaaacaggcaaaaaaacctctcctgtcactgagctgccaccacccaaccaccacctgctgtgggctttgtctcccaagacaaaggacacacagccttatccaatattcaacattacttataaaaacgctgatcagaagaaataccaagtatttcctcagagactgttatatcctttcatcggcaacaagagatgaaatacaacagagtgaatatcaaagaaggcggcaggagccaccgtggcaccatcaccgggcagtgcagtgcccaactgccgttttctgagcacgcataggaagccgtcagtcacatgtaataaaccaaaacctggtacagttatattatggatccccgggtaccgcgatcgctcgcgacctgcaggcataaagccgtcagtgtccgcataaagaaccacccataatacccataatagctgtttgccatcgctaccttaggaccgttatagttaaccggtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggccggccgagctcggtagcaattcccgaggctgtagccgacgatggtgccaccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacataagggactgacctacccggggatcctctagagccatggtgtttaaacgttaactgtaattgtaaatagtaattgtaatgttgtttgttgtttgttgttgttggtaattgttgtaaaaatactcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttcttttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttcttttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttcaagcttgcggccgctcgctaccttaggaccgttatagttaattaccctgttatccctattaattaagagctcgctaccttaagagagaccggtgaattcgcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagctggaagatttgatggatcctcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatctgtttaaacgttaacggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaatcgttgcggttctgtcagttccaaacgtaaaacggcttgtcccgcgtcatcggcgggggtcataacgtgactcccttaattctccgctcatgatcaagcttgcggccgcggcgcgcctctagaaaaccaatatatttccaaatgaaaaaaaaatctgataaaaagttgactttaaaaaaggtatcaataaatgtatgcatttctcactagccttaaactctgcatgaagtgtttgatgagcagatgaagacaacatcatttctagtttcagaaataataacagcatcaaaaccgcagctgtaactccactgagctcacgttaagttttgatgtgtgaatatctgacagaactgacataatgagcactgcaaggatatcagacaagtcaaaatgaagacagacaaaagtattttttaatataaaaatggtctttatttcttcaatacaaggtaaactactattgcagtttaagaccaacacaaaagttggacagcaaattgcttaacagtctcctaaaggctgaaaaaaaggaacccatgaaagctaaaagttatgcagtatttcaagtataacatctaaaaatgatgaaacgatccctaaaggtagagattaactaagtacttctgctgaaaatgtattaaaatccgcagttgctaggataccatcttaccttgttgagaaatacaggtctccggcaacgcaacattcagcagactctttggcctgctggaatcaggaaactgcttactatatacacatataaatcctttggagttgggcattctgagagacatccatttcctgacattttgcagtgcaactctgcattccaactcagacaagctcccatgctgtatttcaaagccatttcttgaatagtttacccagacatccttgtgcaaattgggaatgaggaaatgcaatggtacaggaagacaatacagccttatgtttagaaagtcagcagcgctggtaatcttcataaaaatgtaactgttttccaaataggaatgtatttcacttgtaaaacacctggtcctttttatattacttttttttttttttaaggacacctgcactaatttgcaatcacttgtatttataaaagcacacgcactcctcattttcttacatttgaagatcagcagaatgtctctttcataatgtaataatcatatgcacagtttaaaatattttctattacaaaatacagtacacaagagggtgaggccaaagtctattacttgaatatattccaaagtgtcagcactgggggtgtaaaattacattacatggtatgaataggcggaattcttttacaactgaaatgctcgatttcattgggatcaaaggtaagtactgtttactatcttcaagagacttcaatcaagtcggtgtatttccaaagaagcttaaaagattgaagcacagacacaggccacaccagagcctacacctgctgcaataagtggtgctatagaaaggattcaggaactaacaagtgcataatttacaaatagagatgctttatcatactttgcccaacatgggaaaaaagacatcccatgagaatatccaactgaggaacttctctgtttcatagtaactcatctactactgctaagatggtttgaaaagtacccagcaggtgagatatgttcgggaggtggctgtgtggcagcgtgtcccaacacgacacaaagcaccccacccctatctgcaatgctcactgcaaggcagtgccgtaaacagctgcaacaggcatcacttctgcataaatgctgtgactcgttagcatgctgcaactgtgtttaaaacctatgcactccgttaccaaaataatttaagtcccaaataaatccatgcagcttgcttcctatgccaacatattttagaaagtattcattcttctttaagaatatgcacgtggatctacacttcctgggatctgaagcgatttatacctcagttgcagaagcagtttagtgtcctggatctgggaaggcagcagcaaacgtgcccgttttacatttgaacccatgtgacaacccgccttactgagcatcgctctaggaaatttaaggctgtatccttacaacacaagaaccaacgacagactgcatataaaattctataaataaaaataggagtgaagtctgtttgacctgtacacacagagcatagagataaaaaaaaaaggaaatcaggaattacgtatttctataaatgccatatatttttactagaaacacagatgacaagtatatacaacatgtaaatccgaagttatcaacatgttaactaggaaaacatttacaagcatttgggtatgcaactagatcatcaggtaaaaaatcccattagaaaaatctaagcctcgccagtttcaaaggaaaaaaaccagagaacgctcactacttcaaaggaaaaaaaataaagcatcaagctggcctaaacttaataaggtatctcatgtaacaacagctatccaagctttcaagccacactataaataaaaacctcaagttccgatcaacgttttccataatgcaatcagaaccaaaggcattggcacagaaagcaaaaagggaatgaaagaaaagggctgtacagtttccaaaaggttcttcttttgaagaaatgtttctgacctgtcaaaacatacagtccagtagaaattttactaagaaaaaagaacaccttacttaaaaaaaaaaaacaacaaaaaaaacaggcaaaaaaacctctcctgtcactgagctgccaccacccaaccaccacctgctgtgggctttgtctcccaagacaaaggacacacagccttatccaatattcaacattacttataaaaacgctgatcagaagaaataccaagtatttcctcagagactgttatatcctttcatcggcaacaagagatgaaatacaacagagtgaatatcaaagaaggcggcaggagccaccgtggcaccatcaccgggcagtgcagtgcccaactgccgttttctgagcacgcataggaagccgtcagtcacatgtaataaaccaaaacctggtacagttatattatggatcctacgtactcgagaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtcc

(19) United States
(12) Patent Application Publication
(10) Pub. No.: US 2008/0092252 A1
(43) Pub. Date: Apr. 17, 2008

(54) Enhanced Expression

(75) Inventors: Bruno Philippe Angelo Cammue, Alsemberg (BE); Miguel Francesco Coleta De Bolle, Baarle-Nassau (NL); Katleen Butaye, Hove (BE)

Correspondence Address: MICHAEL BEST & FRIEDRICH LLP, ONE SOUTH PINCKNEY STREET, P O BOX 1806, MADISON, WI, 53701, US

(73) Assignee: PLANT BIOSCIENCE LIMITED, NORWICH NORFOLK (GB)

(21) Appl. No.: 10/581,472

(22) PCT Filed: Nov. 30, 2004
(86) PCT No.: PCT/GB04/05058

§ 371 Date: Nov. 2, 2006

(30) Foreign Application Priority Data
Dec. 2, 2003 (GB) 0327919.7

(51) Int. Cl. A01H 1/00; C07H 21/04; C12N 15/00; C12P 21/04

(52) U.S. Cl. 800/278; 435/320.1; 435/468; 435/69.1; 536/22.1

(57) ABSTRACT

Disclosed herein are methods and means of achieving enhanced expression of a target nucleotide sequence in a transgenic organism, which methods comprise the steps of: (i) providing an organism in which post-transcriptional gene silencing (PTGS) is suppressed, (ii) associating said target nucleotide sequence with one or more heterologous Matrix Attachment Region (MARs), and (iii) causing or permitting expression from the target nucleotide sequence in the organism. Unexpectedly, the MARs do not merely relieve gene silencing, but can actually lead to expression levels higher than can be achieved in wild-type organisms and higher than expression levels in organisms in which PTGS is suppressed but where the MARs are not employed.

### TECHNICAL FIELD

The present invention relates generally to methods and materials for boosting gene expression.

### BACKGROUND ART

In plants, post-transcriptional gene silencing (PTGS) is manifested as the reduction in steady-state levels of specific RNAs after introduction of homologous sequences in the plant genome. This reduction is caused by an increased turnover of target RNA species, with the transcription level of the corresponding genes remaining unaffected.

It is known that suppressing PTGS e.g. by mutating or otherwise impairing the function of the mechanistic genes which support it will increase the expression of silenced genes, back to non-silenced levels.

For example the SGS2 and SGS3 genes were found by mutation of a silenced A. thaliana plant line containing nptII/p35S/uidA/tRBC (Elmayan, et al. 1998). GUS activity was restored after mutation. The SDE1 and SDE3 genes were found by mutation of a silenced plant line containing p35S/PVX:GFP amplicon and p35S/GFP (Dalmay, et al. 2000b). GFP fluorescence was restored after mutation.

Nevertheless, it will be appreciated that methods of increasing the expression of genes over and above those achieved even in such silencing defective contexts would provide a contribution to the art.

### DISCLOSURE OF THE INVENTION

The present inventors have discovered that expression of a target gene in a PTGS-suppressed background can be additionally enhanced by the use of Matrix Attachment Regions (MARs). MARs are non-transcribed regions in eukaryotic genomes that are attached to the proteinaceous matrix in the nucleus (reviewed by Holmes-Davis & Comai, 1998; Allen et al., 2000).

It has been hypothesized in the art that MARs may have a role in shielding sequences from gene silencing. In some cases, transgene expression dropped when MARs were removed from homozygous, high-expressing transgenic tobacco lines (Mlynarova et al., 2003 The Plant Cell: 15, 2203-2217). However, when MARs were used to flank vector constructs for transformation of Arabidopsis thaliana, no PTGS-shielding effect was observed in populations of hemizygous, primary transformants (De Bolle & Butaye et al. (2003)).

Irrespective of the results above, it was not known or expected that MARs could further enhance expression in contexts in which silencing was impaired by a different mechanism.

Briefly, the present inventors demonstrated that the influence of MARs on the level and the variability of gene expression in Arabidopsis thaliana differed significantly between wild-type plants and various A. thaliana mutants impaired in the RNA silencing mechanism, with much greater levels of expression being shown by the latter. In one embodiment of the invention it was estimated that GUS expression was enhanced to the extent that the protein accumulated to roughly 10% of the total soluble proteins in the vegetative tissues of transgenic plants.

Particular aspects of, and definitions used in, the invention will now be discussed in more detail.

In general the invention provides a method of producing a transgenic organism in which a target nucleotide sequence is expressed at an enhanced level, the method comprising the steps of:
(i) providing an organism in which PTGS has been suppressed (which suppression may be pre-existing, or may require the step of suppressing PTGS in the organism e.g. using the methods discussed below),
(ii) associating said target nucleotide sequence with one or more heterologous Matrix Attachment Region (MARs), and optionally:
(iii) causing or permitting expression from the target nucleotide sequence in the organism.

Thus, for example, the invention provides a method of achieving enhanced expression of a heterologous target nucleotide sequence in an organism which is deficient in one or more genes required to support PTGS, which method comprises the steps of associating said target nucleotide sequence with one or more MARs. In one embodiment, the or each of the MARs may be introduced to and associated at random with a pre-existing gene present in the genome of the organism (e.g. to positions flanking it).

The target nucleotide sequence may be one which is endogenous, but is operably linked to a strong, heterologous promoter or enhancer sequence. Such methods may involve:
(i) providing an organism in which PTGS has been, or is suppressed (as discussed herein),
(iia) operably linking said target nucleotide sequence with a heterologous strong promoter or enhancer sequence, and
(iib) associating said target nucleotide sequence with one or more MARs.

Such methods could be performed analogously to existing studies where e.g. the 35S-promoter is introduced at random into a genome to alter the expression of neighbouring endogenous genes, “endogenes”; or e.g. activation-tagging in which enhancers of the p35S are randomly inserted into a genome to activate/increase the expression of endogenes for selection of altered phenotypes (Weigel, D., et al. (2000) Activation tagging in Arabidopsis. Plant Physiol., 122: 1003-13).

In one embodiment this may be carried out as follows:
(i) providing an organism in which PTGS has been, or is suppressed (as discussed herein),
(iia) providing a target nucleic acid construct comprising (a) a promoter, and (b) one or more Matrix Attachment Regions (MARs) associated therewith,
(iib)introducing said target construct into a cell of the organism, such that the promoter becomes operably linked to an endogenous target nucleotide sequence.

In another, preferred embodiment, the target nucleotide sequence and promoter will both be heterologous to the organism. Thus this aspect of the invention provides a method of producing a transgenic organism in which a heterologous target nucleotide sequence is expressed at an enhanced level, the method comprising the steps of:
(i) providing an organism in which PTGS has been suppressed,
(iia) providing a target nucleic acid construct comprising (a) an expression cassette including the target nucleotide sequence operably linked to a promoter, and (b) one or more Matrix Attachment Regions (MARs) associated therewith,
(iib) introducing said target construct into a cell of the organism.

In principle the steps of the method may be carried out in any order i.e. the PTGS may be suppressed after introduction of the construct. Thus the invention provides the steps of:
(i) providing an organism,
(iia) associating the target nucleotide sequence with one or more MARs in a cell of the organism as discussed above,
(iib) suppressing PTGS in the organism e.g. using the methods discussed below (gene mutation or so on).

However preferably the organism will be one in which PTGS is already suppressed.

In preferred embodiments, the invention is used to enhance expression, particularly the level of translation, of a nucleic acid in a cell, particularly a plant cell. Expression may be enhanced, for instance, by at least about 25-50%, preferably about 50-100%, or more. In certain preferred embodiments at least 5, 10, 15, 20, 25, or 30-fold enhancements of expression may be achieved.

Some particular preferred embodiments will now be discussed.

### PTGS Suppression

Preferably the organism is one which is deficient in one or more genes required to support PTGS e.g. a plant deficient in one or more of the following:
1) SGS2/SDE1: RdRp (Dalmay et al., 2000, Mourrain et al., 2000)
2) SGS3: coiled coil protein with unknown function (Mourrain et al., 2000)
3) SDE3: RNA helicase (Dalmay et al., 2001)
4) AGO1: PAZ-domain protein (Fagard et al., 2000)
5) WEX: RNAse D (Glazov et al., 2003)

By “deficient” is meant that the activity of the gene (or encoded protein) is impaired. Preferably the gene may be mutated (e.g. a lesion introduced) or otherwise deleted or knocked out. It will be appreciated that such PTGS suppressed organisms may not be entirely PTGS-deficient. The degree of PTGS impairment or deficiency may be assessed using conventional methods e.g. by monitoring the short RNA species (around 25 nt e.g. about 21-23 nt RNA) associated with PTGS, or by monitoring mRNA and\or expressed protein (Northern or Western Blots or a reporter gene such as GFP) the existence and severity of PTGS can be assessed (see Hamilton and Baulcombe 1999).

Other means of generally suppressing or silencing PTGS supporting genes will be known to those skilled in the art, and include the use of viral suppressors of GS such as HC-Pro (Anandalakshmi et al., 1998) and RNAi, which is widely used as a technique to suppress certain target genes and to create ‘knock-outs’ e.g. in functional genomic programs.

As is well known to those skilled in the art, RNAi can be initiated using hairpin constructs that are designed to trigger PTGS of the target gene, based on homology of sequences (Helliwell and Waterhouse 2003). This technique could therefore also be used to silence genes that play a role in PTGS (e.g. SGS2) in plant lines in which the invention is to be applied. RNAi may be achieved by use of an appropriate vector e.g. a vector comprising part of a nucleic acid sequence encoding a PTGS mechanistic gene, which is suitable for triggering RNAi in the cell. For example the vector may comprise a nucleic acid sequence in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs (see for example Myers (2003) Nature Biotechnology 21:324-328).

Mars optionally only 1 MAR may be associated with the expression cassette, in which case preferably it will be 5′ of the cassette (see e.g. Scöffl e.a. 1993, Transgenic Res. 2, 93-100; van der Geest e.a. 1994, Plant J. 6, 413-423).

Preferably however 2 MARs will be used, which may be the same or different, and which may be from the same or different sources, and these will flank the expression cassette or target nucleotide sequence.

In preferred embodiments the or each MARs will be less than 500, preferably less than 200, and optionally less than 150, 100, or 50 nucleotides upstream of the promoter or downstream of the terminator.

The present invention relates to the use of any MAR origin (e.g. animal, plant, yeast) although preferred examples include that from the the chicken lysozyme gene, or from plants such as petunia and tobacco. Other MARs are reviewed in Holmes-Davis and Comai (1998) and Allen, et. al (2000).

### Organism

The invention may be applied to any organism in which PTGS can be suppressed, particularly eukaryotic organisms including yeasts, fungi, algae, higher plants. Transformed organisms of the present invention will be non-human. Preferably the organism is a higher plant e.g. Arabidopsis thaliana.

### Promoter

Preferably the promoter used to drive the gene of interest will be a strong promoter. Examples of strong promoters for use in plants include:
(1) p35S: Odell et al., 1985
(2) Cassava Vein Mosaic Virus promoter, pCAS, Verdaguer et al., 1996
(3) Promoter of the small subunit of ribulose biphosphate carboxylase, pRbcS: Outchkourov et al., 2003. However other strong promoters include pUbi (for moncots and dicots) and pActin.

### Choice of Target Genes to Enhance

As discussed above, the target gene may be a transgene or an endogene.

Genes of interest include those encoding agronomic traits, insect resistance, disease resistance, herbicide resistance, sterility , grain characteristics, and the like. The genes may be involved in metabolism of oil, starch, carbohydrates, nutrients, etc. Thus genes or traits of interest include, but are not limited to, environmental- or stress-related traits, disease-related traits, and traits affecting agronomic performance. Target sequences also include genes responsible for the synthesis of proteins, peptides, fatty acids, lipids, waxes, oils, starches, sugars, carbohydrates, flavors, odors, toxins, carotenoids, hormones, polymers, flavonoids, storage proteins, phenolic acids, alkaloids, lignins, tannins, celluloses, glycoproteins, glycolipids, etc.

Most preferably the targeted genes in monocots and/or dicots may include those encoding enzymes responsible for oil production in plants such as rape, sunflower, soya bean and maize; enzymes involved in starch synthesis in plants such as potato, maize, cereals; enzymes which synthesise, or proteins which are themselves, natural medicaments such as pharmaceuticals or veterinary products.

Heterologous nucleic acids may encode, inter alia, genes of bacterial, fungal, plant or animal origin. The polypeptides may be utilised in planta (to modify the characteristics of the plant e.g. with respect to pest susceptibility, vigour, tissue differentiation, fertility, nutritional value etc.) or the plant may be an intermediate for producing the polypeptides which can be purified therefrom for use elsewhere. Such proteins include, but are not limited to retinoblastoma protein, p53, angiostatin, and leptin. Likewise, the methods of the invention can be used to produce mammalian regulatory proteins. Other sequences of interest include proteins, hormones, growth factors, cytokines, serum albumin, haemoglobin, collagen, etc.

Thus the target gene or nucleotide sequence preferably encodes a target protein which is : an insect resistance protein; a disease resistance protein; a herbicide resistance protein; a mammalian protein.

### Constructs & Organisms

Preferably the target construct is a vector, and preferably it comprises border sequences which permit the transfer and integration of the expression cassette and MARs into the organism genome.

Preferably the construct is a plant binary vector. Preferably the binary transformation vector is based on pPZP (Hajdukiewicz, et al. 1994). Other example constructs include pBin19 (see Frisch, D. A., L. W. Harris-Haller, et al. (1995). “Complete Sequence of the binary vector Bin 19.” Plant Molecular Biology 27: 405-409).

Preferably the construct used is substantially similar to pFAJ3163 shown in FIG. 1 i.e. comprises the depicted features of that vector (or equivalents as described herein) in the recited order, and the gene of interest in place of the the β-glucuronidase reporter gene (uidA). In embodiments in which endogenes are being activated by a promoter or enhancer element, the coding region of the construct may be absent.

In one aspect the invention may further comprise the step of regenerating a plant from a transformed plant cell.

Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809). If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984; the floral dip method of Clough and Bent, 1998), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11. Ti-plasmids, particularly binary vectors, are discussed in more detail below.

Agrobacterium transformation is widely used by those skilled in the art to transform dicotyledonous species. However there has also been considerable success in the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice.

It will be apparent to the skilled person that the particular choice of a transformation system to introduce nucleic acid into plant cells is not essential to or a limitation of the invention, nor is the choice of technique for plant regeneration. In experiments performed by the inventors, the enhanced expression effect is seen in a variety of integration patterns of the T-DNA.

Thus various aspects of the present invention provide a method of transforming a plant cell involving introduction of a construct of the invention into a plant tissue (e.g. a plant cell) and causing or allowing recombination between the vector and the plant cell genome to introduce a nucleic acid according to the present invention into the genome. This may be done so as to effect transient expression. Alternatively, following transformation of plant tissue, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Regenerated plants or parts thereof may be used to provide clones, seed, selfed or hybrid progeny and descendants (e.g. F1 and F2 descendants), cuttings (e.g. edible parts) etc.

The invention further provides a transgenic organism (for example obtained or obtainable by a method described herein) in which an heterologous target nucleotide sequence is expressed at an enhanced level,
wherein the organism is deficient in one or more genes required to support PTGS,
which organism includes in its genome (a) an expression cassette including the target nucleotide sequence operably linked to a promoter, and (b) one or more heterologous Matrix Attachment Regions (MARs) associated therewith.

The invention further comprises a method for generating a target protein, which method comprises the steps of performing a method (or using an organism) as described above, and optionally harvesting, at least, a tissue in which the target protein has been expressed and isolating the target protein from the tissue.

### Definitions

“Matrix attachment region” (MARs) are non coding DNA sequences that are thought to mediate the binding of chromatin to the proteinaceous nuclear matrix, thereby creating chromatin domains as topologically isolated units of gene regulation.

The term “heterologous” is used broadly below to indicate that the gene/sequence of nucleotides in question have been introduced into the cells in question (e.g. of a plant or an ancestor thereof) using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a cell may be non-naturally occurring in cells of that type, variety or species. Thus the heterologous nucleic acid may comprise a coding sequence of, or derived from, a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

“Gene” unless context demands otherwise refers to any nucleic acid encoding genetic information for translation into a peptide, polypeptide or protein.

“Vector” is defined to include, inter alia, any plasmid, cosmid, phage, viral or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication). The constructs used will be wholly or partially synthetic. In particular they are recombinant in that nucleic acid sequences which are not found together in nature (do not run contiguously) have been ligated or otherwise combined artificially. Unless specified otherwise a vector according to the present invention need not include a promoter or other regulatory sequence, particularly if the vector is to be used to introduce the nucleic acid into cells for recombination into the genome.

“Binary Vector”: as is well known to those skilled in the art, a binary vector system includes (a) border sequences which permit the transfer of a desired nucleotide sequence into a plant cell genome; (b) desired nucleotide sequence itself, which will generally comprise an expression cassette of (i) a plant active promoter, operably linked to (ii) the target sequence and\or enhancer as appropriate. The desired nucleotide sequence is situated between the border sequences and is capable of being inserted into a plant genome under appropriate conditions. The binary vector system will generally require other sequence (derived from A. tumefaciens) to effect the integration. Generally this may be achieved by use of so called “agro-infiltration” which uses Agrobacterium-mediated transient transformation. Briefly, this technique is based on the property of Agrobacterium tumefaciens to transfer a portion of its DNA (“T-DNA”) into a host cell where it may become integrated into nuclear DNA. The T-DNA is defined by left and right border sequences which are around 21-23 nucleotides in length. The infiltration may be achieved e.g. by syringe (in leaves) or vacuum (whole plants). In the present invention the border sequences will generally be included around the desired nucleotide sequence (the T-DNA) with the one or more vectors being introduced into the plant material by agro-infiltration.

“Expression cassette” refers to a situation in which a nucleic acid is under the control of, and operably linked to, an appropriate promoter or other regulatory elements for transcription in a host cell such as a microbial or plant cell.

A “promoter” is a sequence of nucleotides from which transcription may be initiated of DNA operably linked downstream (i.e. in the 31 direction on the sense strand of double-stranded DNA).

“Operably linked” means joined as part of the same nucleic acid molecule, suitably positioned and oriented for transcription to be initiated from the promoter.

It will be appreciated that where a nucleotide sequence (e.g. a specific MAR, gene, polypeptide, promoter etc.) is referred to or exemplified herein, the invention should not be taken to be limited to use of the recited sequence, but also embraces use of a variants of any of these sequences. A variant sequence will be identical to all or part of the sequence discussed and share the requisite activity, which activity can be confirmed using the methods disclosed or otherwise referred to herein or known to those skilled in the art. Generally speaking, wherever the term is used herein, variants may be
(i) naturally occurring homologous variants of the relevant sequence;
(ii) artificially generated variants (derivatives) which can be prepared by the skilled person in the light of the present disclosure, for instance by site directed or random mutagenesis, or by direct synthesis. Preferably any variant sequence shares at least about 75%, or 80% identity, most preferably at least about 90%, 95%, 96%, 97%, 98% or 99% identity with that specifically referred to. Similarity or homology in the case of variants is preferably established via sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA. Homology may also be assessed by use of a probing methodology (Sambrook et al., 1989). One common formula for calculating the stringency conditions required to achieve hybridization between nucleic acid molecules of a specified sequence homology is: Tₘ=81.5° C.+16.6 Log [Na+]+0.41 (% G+C)−0.63 (% formamide)−600/#bp in duplex. As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1 Schematic representation of the T-DNA region of plant transformation vectors pFAJ3160 and pFAJ3163. Not to scale. UidA: β-glucuronidase coding region; pat: phosphinothricin acetyltransferase coding region; pNOS: nopaline synthase promoter; p35S: cauliflower mosaic virus 35S promoter; tOCS: octopine synthase terminator; tNOS: nopaline synthase terminator; ChilMAR: chicken lysozyme MAR; RB and LB: right and left T-DNA border, respectively.

FIG. 2 GUS activity is expressed in units GUS (nmoles 4-methylumbelliferone per min per mg total soluble protein) in first generation transgenic A. thaliana wild-type, sgs2 and sgs3 background transformed with pFAJ3160 and pFAJ3163.

FIG. 3 SDS-PAGE analysis of total protein extracts (2μg/lane) from sgs2 mutants transformed with pFAJ3163 (lanes 1 and 2); total protein extracts (2 μg/lane) from non-transgenic plants (lane 3); 500 ng bovine serum albumin (lane 4); partially purified β-glucuronidase (lane 5). The position of GUS is indicated by the arrow to the right. The position of molecular weight reference proteins is indicated by arrows to the left.

### SEQUENCES

Where a DNA sequence is specified, unless context requires otherwise, use of the RNA equivalent, with U substituted for T where it occurs, is encompassed.
Sequence Annex 1: Chicken lysozyme MAR
Sequence Annex 2: pFAJ3160
Sequence Annex 3: pFAJ3163

### EXAMPLES

### Materials and Methods

Briefly, a set of transformation vectors was constructed without and with MARs flanking the genes of interest. To quantify transgene expression the β-glucuronidase reporter gene (uidA) driven by the 35S promoter of Cauliflower Mosaic Virus (p35S) was used. For each plant transformation vector A. thaliana populations consisting of at least 30 primary transformants were obtained. The activity of the β-glucuronidase (GUS) enzyme in leaf extracts was measured and statistically evaluated.

### Plant Transformation Vectors

All plant transformation vectors were constructed using the modular vector system as fully described in Goderis & De Bolle et al. (2002). pFAJ3160 and pFAJ3163 were assembled as previously described in De Bolle & Butaye et al. (2003) (see Sequence Annex).

### Mutants sgs2 and sgs3 mutants as described in Elmayan et al. (1998) and Mourrain et al. (2000). Seeds of the mutants were provided by Hervé Vaucheret, INRA Versailles.

### Plant Transformation

All plant transformation vectors were introduced in Agrobacterium tumefaciens GV3101 (pMP90) by electroporation. The A. tumefaciens strains with the binary vectors were used to transform A. thaliana wild-type and mutant plants using the floral dip transformation method as described by Clough & Bent (1998). Transgenic plants were selected based on resistance against phosphinotricin and further grown as described by De Bolle & Butaye et al. (2003).

### Enzyme Assays

β-Glucuronidase (GUS) activity was measured fluorometrically using 4-methylumbelliferyl glucuronide as a substrate and 4-methylubmelliferon as a standard according to Jefferson (1987). Total protein was determined by the method of Bradford (1976) using bovine serum albumin as a standard.

### SDS-PAGE

Total leaf extracts and GUS standard (Sigma-Aldrich) were separated on a 12.5% SDS-PAGE and visualized by staining with Coomassie brilliant blue R250.

### Results

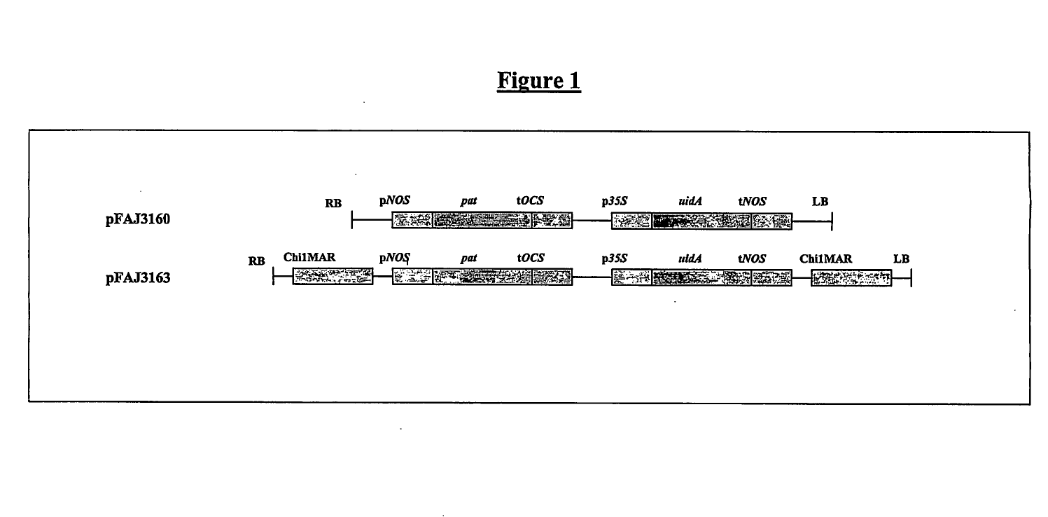

The A element that flanks the chicken lysozyme gene (Phi-Van et al., 1990; chilMAR) has been shown to reduce transgene expression variability in tobacco (Mlynárová et al., 1994). To test the effect of chilMAR on transgene expression in A. thaliana plant transformation vectors without and with chilMARs flanking the T-DNA region were constructed, pFAJ3160 and pFAJ3163 respectively (FIG. 1).

### ChilMAR in ColO

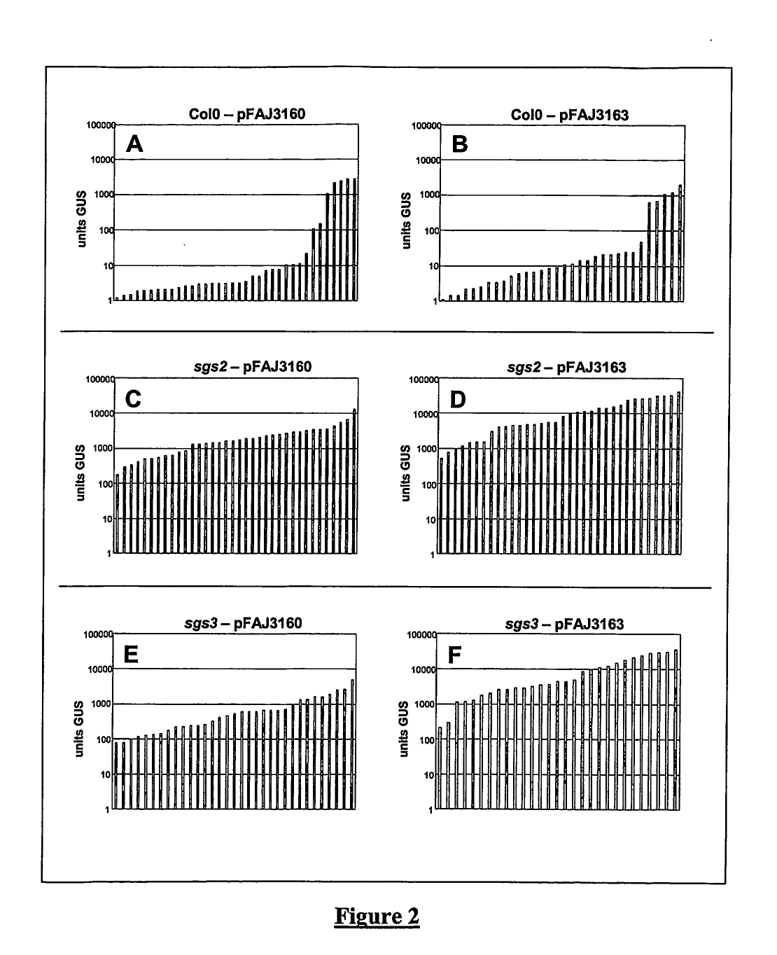

Transformation of wild-type A. thaliana plants with pFAJ3160 yielded an average GUS activity of 320 units (Table 1). The population of primary transformants consisted of about 80% low GUS expressing primary transformants (<50 units GUS) and about 20% high GUS expressing primary transformants (>100 units GUS), a bimodal distribution typical for p35S-driven expression (Elmayan & Vaucheret, 1996; De Bolle & Butaye et al., 2003; FIG. 2A). To test the influence of chilMARs on transgene expression, wild-type plants were transformed with pFAJ3163. This resulted in a pattern of GUS activity similar to the one obtained with pFAJ3160 (Table 1; FIG. 2B). It was concluded that chilMARs have no significant influence on the level of transgene expression or on the variability of transgene expression in populations of first generation wild-type A. thaliana transformants (De Bolle & Butaye et al., 2003).

TABLE 1
GUS activity in first generation transgenic Arabidopsis
thaliana wild-type, sgs2 and sgs3 background transformed with
pFAJ3160 and pFAJ3163.

| | GUS activity^{a} | | | |
|---|---|---|---|---|
| | pFAJ3160 (−MAR) | | pFAJ3163 (+MAR) | |
| Background | No^{b} | Mean ± S.E.^{c} | No^{b} | Mean ± S.E.^{c} |
| Col0 | 36 | 320 ± 135 | 36 | 186 ± 81 |
| sgs2 | 36 | 2280 ± 399 | 34 | 11 237 ± 1839 |
| sgs3 | 33 | 830 ± 177 | 30 | 9994 ± 2006 |

^{a}GUS activity is expressed in units GUS (nmoles 4-methylumbelliferone per min per mg total soluble protein).
^{b}Number of primary transformants analyzed.
^{c}S.E., Standard error.

### ChilMAR in sgs2

In a further attempt to elevate and level off transgene expression, A. thaliana sgs2 mutants (Elmayan, et al., 1998) were used as the recipient for transformation instead of wild-type plants. SGS2 encodes an RNA dependent RNA polymerase, which is presumed to play a key role in RNA silencing of transgenes (Mourrain, et al. 2000). Using this mutant background for transformation with pFAJ3160, average GUS activity in primary transformants increased almost 8-fold compared to wild-type plants (Table 1). The increase in average GUS activity at the population level was not due to an increase in activity of the high-expressing individuals but rather to a reduction of the incidence of individuals with low expression. About 80% of the transformants in the wild-type background had a GUS activity below 50 units GUS, whereas all sgs2 transformants had a GUS activity above 180 units GUS (FIG. 2C). Upon transformation of sgs2 mutants with pFAJ3163, chilMARs caused a 5-fold increase in average GUS activity compared to pFAJ3160 in sgs2. Compared to pFAJ3160 in wild-type plants, the chilMARs caused a 40-fold boost of mean GUS activity in sgs2 mutants (Table 1; FIG. 2D).

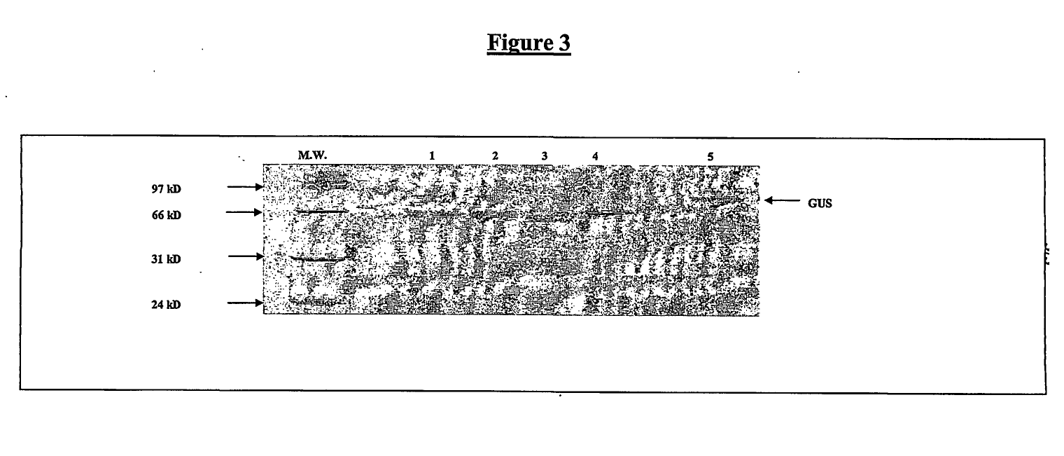

Some of the sgs2 transformants containing chilMAR-flanked transgenes reached extremely high GUS activity levels, up to 41 000 units GUS. Coomassie blue staining of an SDS-PAGE gel revealed a clear band in the total leaf extracts of extremely high GUS expressing sgs2 mutants (FIG. 3, lanes 1 & 2), which is not visible in the total leaf extracts of non-transgenic control plants (FIG. 3, lane 3) and which is situated at the same position in the gel as the GUS standard (FIG. 3, lane 5). By densitometric comparison of the intensities of this band to known amounts of bovine serum albumin (BSA; FIG. 3, lane 4) we estimate that GUS accumulated to roughly 10% of the total soluble protein in the transgenic sgs2 plants.

### ChilMAR in sgs3

SGS3 plays a yet unknown key role in the RNA silencing mechanism and shows no similarity with any known or putative protein (Mourrain, et al., 2000). Using sgs3 mutants for transformation with pFAJ3160, the average GUS activity was increased 2,5 fold in comparison the wild-type background (Table 1, FIG. 2E). Transformation of sgs3 plants with pFAJ3163 yielded a 30-fold increase of the average GUS activity in comparison to wild-type plants transformed with pFAJ3160.

### References

Allen, G. C., Spiker, S., Thompson, W. F. (2000). Use of matrix attachment regions (MARs) to minimize transgene silencing. Plant Mol. Biol. 43: 361-376.

Anandalakshmi, R., Pruss, G. J., Ge, X., Marathe, R., Mallory, A. C., Smith, T. H., Vance, V. B. (1998) A viral suppressor of gene silencing in plants Proc Natl Acad Sci USA, 95, 13079-1384.

Bradford, M. M. (1976). A rapid and sensitive method for quantification of microgram quantities utilizing the principle of protein-dye binding, Anal. Biochem. 72: 248-254.

Clough, S. J. & Bent, A. F. (1998). Floral dip: a simplified method for Agrobacterium-mediated transformation of Arabidopsis thaliana. Plant J. 16: 735-43.

Dalmay, T., Hamilton, A., Rudd, S., Angell, S. and Baulcombe, D. C. (2000) An RNA-dependent RNA polymerase gene in Arabidopsis is required for posttranscriptional gene silencing mediated by a transgene but not by a virus. Cell, 101, 543-553.

Dalmay, T., Horsefield, R., Braunstein, T. H. and Baulcombe D. C. (2001) SDE3 encodes an RNA helicase required for post-transcriptional gene silencing in Arabidopsis. EMBO J. 20, 2069-2078.

De Bolle, M. F. C., Butaye, K. M. J., Coucke, W. J. W., Goderis, I. J. W. M., Wouters, F. J., van Boxel, N., Broekaert, W. F., Cammue, B. P. A. (2003). Analysis of the influence of promoter elements and a matrix attachment region on the inter-individual variation of transgene expression in populations of Arabidopsis thaliana. Plant Science 165: 169-179.

Elmayan, T. & Vaucheret, H. (1996). Expression of single copies of a strongly expressed 35S transgene can be silenced post-transcriptionally. Plant J. 9: 787-797.

Elmayan, T., Balzergue, S., Béon, F., Bourdon, V., Daubremet, J., Guénet, Y., Mourrain, P., Palauqui, J.-C., Vernhettes, S., Vialle, T., Wostrikoff, K., Vaucheret, H. (1998). Arabidopsis mutants impaired in cosuppression. Plant Cell 10: 1747-1757.

Epinat, J. C., Arnould, S., Chames, P., Rochaix, P., Desfontaines, D., Puzin, O., Patin, A., Zanghellini, A., Paques, F., Lacroix, E. (2003). A novel engineered meganuclease induces homologous recombination in yeast and mamalian cells. Nucleic Acids Res., 31: 2952-2962.

Fagard, M., Boutet, S., Morel, J. B., Bellini, C. and Vaucheret, H. (2000) AGO1, QDE-2, and RDE-1 are related proteins required for post-transcriptional gene silencing in plants, quelling in fungi, and RNA interference in animals. Proc Natl Acad Sci USA, 97, 11650-11654.

Glazov, E., Phillips, K., Budziszewski, G. J., Meins, F., Levin, J. Z. (2003) A gene encoding an RNase D exonuclease-like protein is required for post-transcriptional silencing in Arabidopsis. Plant J. 35, 342-349.

Goderis, I. J. W. M., De Bolle, M. F. C., Francois, I. E. J. A., Wouters, P. F. J., Broekaert, W. F. & Cammue, B. P. A. (2002). A set of modular plant transformation vectors allowing flexible insertion of up to six expression units. Plant Mol. Biol. 50: 17-27.

Hajdukiewicz, P., Svab, Z. and Maliga P. 1994. The small, versatile pPZP family of Agrobacterium binary vectors for plant transformation. Plant Mol. Biol. 25: 989-994.

Hamilton, A. J. and Baulcombe, D. C. (1999) A species of small antisense RNA in posttranscriptional gene silencing in plants. Science, 286, 950-952.

Holmes-Davis, R., Comai, L. (1998). Nuclear matrix attachment regions and plant gene expression. Trends Plant Sci. 3: 91-97.

Jefferson, R. A. (1987). Assaying chimeric genes in plants : the GUS gene fusion system, Plant Mol. Biol. Rep. 5: 387-405.

Mlynárová, L., Loonen, A., Heldens, J., Jansen, R. C., Keizer, P., Stiekema, W. J., Nap, J. P. (1994). Reduced position effect in mature transgenic plants conferred by the chicken lysozyme matrix-associated region. Plant Cell, 6: 417-426.

Mourrain, P., Béclin, C., Elmayan, T., Feuerbach, F., Godon, C., Morel, J.-B., Jouette, D., Lacombe, A.-M., Nikic, S., Picault, N., Rémoué, K., Sanial, M., Vo, T.-A., Vaucheret, H. (2000). Arabidopsis SGS2 and SGS3 genes are required for posttranscriptional gene silencing and natural virus resistance. Cell, 101: 533-542.

Odell, J. T., Nagy, F. and Chua, N. H. (1985) Identification of DNA sequences required for activity of the cauliflower mosaic virus 35S promoter. Nature, 313, 810-812.

Outchkourov, N. S., Peters, J., de Jong, J., Rademakers, W. and Jongsma, M. A. (2003) The promoter-terminator of chrysanthemum rbcS1 directs very high expression levels in plants. Planta, 216, 1003-1012.

Phi-Van, L., von Kries, J. P., Ostertag W. & Stratling, W. H. (1990). The chicken lysozyme 5′ matrix attachment region increases transcription from a heterologous promoter in heterologous cells and dampens position effects on the expression of transfected genes. Mol. Cell Biol. 10: 2302-2307.

Verdaguer, B., de Kochko, A., Beachy, R. N., Fauquet, C. (1996). Isolation and expression in transgenic tobacco and rice plants, of the cassava vein mosaic virus (CVMV) promoter. Plant Mol. Biol. 31, 1129-1139.

Weigel, D., Ahn, J. H., Blazquez, M. A., Borevitz, J. O., Christensen, S. K., Fankhauser, C., Ferrandiz, C., Kardailsky, I., Malancharuvil, E. J., Neff, M. M., Nguyen, J. T., Sato, S., Wang, Z. Y., Xia, Y., Dixon, R. A., Harrison, M. J., Lamb, C. J., Yanofsky, M. F., Chory, J. (2000). Activation tagging in Arabidopsis. Plant Physiol. 122:1003-1013.

Wesley, S. V., Helliwell, C. A., Smith, N. A., Wang, M. B., Rouse, D. T., Liu, Q., Gooding, P. S., Singh, S. P., Abbott, D., Stoutjesdijk, P. A., Robinson, S. P., Gleave, A. P., Green, A. G., Waterhouse, P. M. (2001) Construct design for efficient, effective and high-throughput gene silencing in plants. Plant J. 27, 581-590.

Sequence Annex 1: Chicken lysozyme MAR

aaaccaatatatttccaaatgaaaaaaaaatctgataaaaagttgactttaaaaaaggtatcaataaat
gtatgcatttctcactagccttaaactctgcatgaagtgtttgatgagcagatgaagacaacatcattt
ctagtttcagaaataataacagcatcaaaaccgcagctgtaactccactgagctcacgttaagttttga
tgtgtgaatatctgacagaactgacataatgagcactgcaaggatatcagacaagtcaaaatgaagaca
gacaaaagtattttttaatataaaaatggtctttatttcttcaatacaaggtaaactactattgcagtt
taagaccaacacaaaagttggacagcaaattgcttaacagtctcctaaaggctgaaaaaaaggaaccca
tgaaagctaaaagttatgcagtatttcaagtataacatctaaaaatgatgaaacgatccctaaaggtag
agattaactaagtacttctgctgaaaatgtattaaaatccgcagttgctaggataccatcttaccttgt
tgagaaatacaggtctccggcaacgcaacattcagcagactctttggcctgctggaatcaggaaactgc
ttactatatacacatataaatcctttggagttgggcattctgagagacatccatttcctgacattttgc
agtgcaactctgcattccaactcagacaagctcccatgctgtatttcaaagccatttcttgaatagttt
acccagacatccttgtgcaaattgggaatgaggaaatgcaatggtacaggaagacaatacagccttatg
tttagaaagtcagcagcgctggtaatcttcataaaaatgtaactgttttccaaataggaatgtatttca
cttgtaaaacacctggtcctttttatattacttttttttttttttaaggacacctgcactaatttgcaa
tcacttgtatttataaaagcacacgcactcctcattttcttacatttgaagatcagcagaatgtctctt
tcataatgtaataatcatatgcacagtttaaaatattttctattacaaaatacagtacacaagagggtg
aggccaaagtctattacttgaatatattccaaagtgtcagcactgggggtgtaaaattacattacatgg
tatgaataggcggaattcttttacaactgaaatgctcgatttcattgggatcaaaggtaagtactgttt
actatcttcaagagacttcaatcaagtcggtgtatttccaaagaagcttaaaagattgaagcacagaca
caggccacaccagagcctacacctgctgcaataagtggtgctatagaaaggattcaggaactaacaagt
gcataatttacaaatagagatgctttatcatactttgcccaacatgggaaaaaagacatcccatgagaa
tatccaactgaggaacttctctgtttcatagtaactcatctactactgctaagatggtttgaaaagtac
ccagcaggtgagatatgttcgggaggtggctgtgtggcagcgtgtcccaacacgacacaaagcacccca
cccctatctgcaatgctcactgcaaggcagtgccgtaaacagctgcaacaggcatcacttctgcataaa
tgctgtgactcgttagcatgctgcaactgtgtttaaaacctatgcactccgttaccaaaataatttaag
tcccaaataaatccatgcagcttgcttcctatgccaacatattttagaaagtattcattcttctttaag
aatatgcacgtggatctacacttcctgggatctgaagcgatttatacctcagttgcagaagcagtttag
tgtcctggatctgggaaggcagcagcaaacgtgcccgttttacatttgaacccatgtgacaacccgcct
tactgagcatcgctctaggaaatttaaggctgtatccttacaacacaagaaccaacgacagactgcata
taaaattctataaataaaaataggagtgaagtctgtttgacctgtacacacagagcatagagataaaaa
aaaaaggaaatcaggaattacgtatttctataaatgccatatatttttactagaaacacagatgacaag
tatatacaacatgtaaatccgaagttatcaacatgttaactaggaaaacatttacaagcatttgggtat
gcaactagatcatcaggtaaaaaatcccattagaaaaatctaagcctcgccagtttcaaaggaaaaaaa
ccagagaacgctcactacttcaaaggaaaaaaaataaagcatcaagctggcctaaacttaataaggtat
ctcatgtaacaacagctatccaagctttcaagccacactataaataaaaacctcaagttccgatcaacg
ttttccataatgcaatcagaaccaaaggcattggcacagaaagcaaaaagggaatgaaagaaaagggct
gtacagtttccaaaaggttcttcttttgaagaaatgtttctgacctgtcaaaacatacagtccagtaga
aattttactaagaaaaaagaacaccttacttaaaaaaaaaaaacaacaaaaaaaacaggcaaaaaaacc
tctcctgtcactgagctgccaccacccaaccaccacctgctgtgggctttgtctcccaagacaaaggac
acacagccttatccaatattcaacattacttataaaaacgctgatcagaagaaataccaagtatttcct
cagagactgttatatcctttcatcggcaacaagagatgaaatacaacagagtgaatatcaaagaaggcg
gcaggagccaccgtggcaccatcaccgggcagtgcagtgcccaactgccgttttctgagcacgcatagg
aagccgtcagtcacatgtaataaaccaaaacctggtacagttatattat

Sequence Annex 2: VpFAJ3160: 11169 bp

agtactttgatccaacccctccgctgctatagtgcagtcggcttctgacgttcagtgcagccgtcttct
gaaaacgacatgtcgcacaagtcctaagttacgcgacaggctgccgccctgcccttttcctggcgtttt
cttgtcgcgtgttttagtcgcataaagtagaatacttgcgactagaaccggagacattacgccatgaac
aagagcgccgccgctggcctgctgggctatgcccgcgtcagcaccgacgaccaggacttgaccaaccaa
cgggccgaactgcacgcggccggctgcaccaagctgttttccgagaagatcaccggcaccaggcgcgac
cgcccggagctggccaggatgcttgaccacctacgccctggcgacgttgtgacagtgaccaggctagac
cgcctggcccgcagcacccgcgacctactggacattgccgagcgcatccaggaggccggcgcgggcctg
cgtagcctggcagagccgtgggccgacaccaccacgccggccggccgcatggtgttgaccgtgttcgcc
ggcattgccgagttcgagcgttccctaatcatcgaccgcacccggagcgggcgcgaggccgccaaggcc
cgaggcgtgaagtttggcccccgccctaccctcaccccggcacagatcgcgcacgcccgcgagctgatc
gaccaggaaggccgcaccgtgaaagaggcggctgcactgcttggcgtgcatcgctcgaccctgtaccgc
gcacttgagcgcagcgaggaagtgacgcccaccgaggccaggcggcgcggtgccttccgtgaggacgca
ttgaccgaggccgacgccctggcggccgccgagaatgaacgccaagaggaacaagcatgaaaccgcacc
aggacggccaggacgaaccgtttttcattaccgaagagatcgaggcggagatgatcgcggccgggtacg
tgttcgagccgcccgcgcacgtctcaaccgtgcggctgcatgaaatcctggccggtttgtctgatgcca
agctggcggcctggccggccagcttggccgctgaagaaaccgagcgccgccgtctaaaaaggtgatgtg
tatttgagtaaaacagcttgcgtcatgcggtcgctgcgtatatgatgcgatgagtaaataaacaaatac
gcaaggggaacgcatgaaggttatcgctgtacttaaccagaaaggcgggtcaggcaagacgaccatcgc
aacccatctagcccgcgccctgcaactcgccggggccgatgttctgttagtcgattccgatccccaggg
cagtgcccgcgattgggcggccgtgcgggaagatcaaccgctaaccgttgtcggcatcgaccgcccgac
gattgaccgcgacgtgaaggccatcggccggcgcgacttcgtagtgatcgacggagcgccccaggcggc
ggacttggctgtgtccgcgatcaaggcagccgacttcgtgctgattccggtgcagccaagcccttacga
catatgggccaccgccgacctggtggagctggttaagcagcgcattgaggtcacggatggaaggctaca
agcggcctttgtcgtgtcgcgggcgatcaaaggcacgcgcatcggcggtgaggttgccgaggcgctggc
cgggtacgagctgcccattcttgagtcccgtatcacgcagcgcgtgagctacccaggcactgccgccgc
cggcacaaccgttcttgaatcagaacccgagggcgacgctgcccgcgaggtccaggcgctggccgctga
aattaaatcaaaactcatttgagttaatgaggtaaagagaaaatgagcaaaagcacaaacacgctaagt
gccggccgtccgagcgcacgcagcagcaaggctgcaacgttggccagcctggcagacacgccagccatg
aagcgggtcaactttcagttgccggcggaggatcacaccaagctgaagatgtacgcggtacgccaaggc
aagaccattaccgagctgctatctgaatacatcgcgcagctaccagagtaaatgagcaaatgaataaat
gagtagatgaattttagcggctaaaggaggcggcatggaaaatcaagaacaaccaggcaccgacgccgt
ggaatgccccatgtgtggaggaacgggcggttggccaggcgtaagcggctgggttgtctgccggccctg
caatggcactggaacccccaagcccgaggaatcggcgtgacggtcgcaaaccatccggcccggtacaaa
tcggcgcggcgctgggtgatgacctggtggagaagttgaaggccgcgcaggccgcccagcggcaacgca
tcgaggcagaagcacgccccggtgaatcgtggcaagcggccgctgatcgaatccgcaaagaatcccggc
aaccgccggcagccggtgcgccgtcgattaggaagccgcccaagggcgacgagcaaccagattttttcg
ttccgatgctctatgacgtgggcacccgcgatagtcgcagcatcatggacgtggccgttttccgtctgt
cgaagcgtgaccgacgagctggcgaggtgatccgctacgagcttccagacgggcacgtagaggtttccg
cagggccggccggcatggccagtgtgtgggattacgacctggtactgatggcggtttcccatctaaccg
aatccatgaaccgataccgggaagggaagggagacaagcccggccgcgtgttccgtccacacgttgcgg
acgtactcaagttctgccggcgagccgatggcggaaagcagaaagacgacctggtagaaacctgcattc
ggttaaacaccacgcacgttgccatgcagcgtacgaagaaggccaagaacggccgcctggtgacggtat
ccgagggtgaagccttgattagccgctacaagatcgtaaagagcgaaaccgggcggccggagtacatcg
agatcgagctagctgattggatgtaccgcgagatcacagaaggcaagaacccggacgtgctgacggttc
accccgattactttttgatcgatcccggcatcggccgttttctctaccgcctggcacgccgcgccgcag
gcaaggcagaagccagatggttgttcaagacgatctacgaacgcagtggcagcgccggagagttcaaga
agttctgtttcaccgtgcgcaagctgatcgggtcaaatgacctgccggagtacgatttgaaggaggagg
cggggcaggctggcccgatcctagtcatgcgctaccgcaacctgatcgagggcgaagcatccgccggtt
cctaatgtacggagcagatgctagggcaaattgccctagcaggggaaaaaggtcgaaaaggtctctttc
ctgtggatagcacgtacattgggaacccaaagccgtacattgggaaccggaacccgtacattgggaacc
caaagccgtacattgggaaccggtcacacatgtaagtgactgatataaaagagaaaaaaggcgattttt
ccgcctaaaactctttaaaacttattaaaactcttaaaacccgcctggcctgtgcataactgtctggcc
agcgcacagccgaagagctgcaaaaagcgcctacccttcggtcgctgcgctccctacgccccgccgctt
cgcgtcggcctatcgcggccgctggccgctcaaaaatggctggcctacggccaggcaatctaccagggc
gcggacaagccgcgccgtcgccactcgaccgccggcgcccacatcaaggcaccctgcctcgcgcgtttc
ggtgatgacggtgaaaacctctgacacatgcagctcccggagacggtcacagcttgtctgtaagcggat
gccgggagcagacaagcccgtcagggcgcgtcagcgggtgttggcgggtgtcggggcgcagccatgacc
cagtcacgtagcgatagcggagtgtatactggcttaactatgcggcatcagagcagattgtactgagag
tgcaccatatgcggtgtgaaataccgcacagatgcgtaaggagaaaataccgcatcaggcgctcttccg
cttcctcgctcactgactcgctgcgctcggtcgttcggctgcggcgagcggtatcagctcactcaaagg
cggtaatacggttatccacagaatcaggggataacgcaggaaagaacatgtgagcaaaaggccagcaaa
aggccaggaaccgtaaaaaggccgcgttgctggcgtttttccataggctccgcccccctgacgagcatc
acaaaaatcgacgctcaagtcagaggtggcgaaacccgacaggactataaagataccaggcgtttcccc
ctggaagctccctcgtgcgctctcctgttccgaccctgccgcttaccggatacctgtccgcctttctcc
cttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgtc
ttgagtccaacccggtaagacacgacttatcgccactggcagcagccactggtaacaggattagcagag
cgaggtatgtaggcggtgctacagagttcttgaagtggtggcctaactacggctacactagaaggacag
tatttggtatctgcgctctgctgaagccagttaccttcggaaaaagagttggtagctcttgatccggca
aacaaaccaccgctggtagcggtggtttttttgtttgcaagcagcagattacgcgcagaaaaaaaggat
ctcaagaagatcctttgatcttttctacggggtctgacgctcagtggaacgaaaactcacgttaaggga
ttttggtcatgcatgatatatctcccaatttgtgtagggcttattatgcacgcttaaaaataataaaag
cagacttgacctgatagtttggctgtgagcaattatgtgcttagtgcatctaatcgcttgagttaacgc
cggcgaagcggcgtcggcttgaacgaatttctagctagacattatttgccgactaccttggtgatctcg
cctttcacgtagtggacaaattcttccaactgatctgcgcgcgaggccaagcgatcttcttcttgtcca
agataagcctgtctagcttcaagtatgacgggctgatactgggccggcaggcgctccattgcccagtcg
gcagcgacatccttcggcgcgattttgccggttactgcgctgtaccaaatgcgggacaacgtaagcact
acatttcgctcatcgccagcccagtcgggcggcgagttccatagcgttaaggtttcatttagcgcctca
aatagatcctgttcaggaaccggatcaaagagttcctccgccgctggacctaccaaggcaacgctatgt
tctcttgcttttgtcagcaagatagccagatcaatgtcgatcgtggctggctcgaagatacctgcaaga
atgtcattgcgctgccattctccaaattgcagttcgcgcttagctggataacgccacggaatgatgtcg
tcgtgcacaacaatggtgacttctacagcgcggagaatctcgctctctccaggggaagccgaagtttcc
aaaaggtcgttgatcaaagctcgccgcgttgtttcatcaagccttacggtcaccgtaaccagcaaatca
atatcactgtgtggcttcaggccgccatccactgcggagccgtacaaatgtacggccagcaacgtcggt
tcgagatggcgctcgatgacgccaactacctctgatagttgagtcgatacttcggcgatcaccgcttcc
cccatgatgtttaactttgttttagggcgactgccctgctgcgtaacatcgttgctgctccataacatc
aaacatcgacccacggcgtaacgcgcttgctgcttggatgcccgaggcatagactgtaccccaaaaaaa
catgtcataacaagaagccatgaaaaccgccactgcgccgttaccaccgctgcgttcggtcaaggttct
ggaccagttgcgtgacggcagttacgctacttgcattacagcttacgaaccgaacgaggcttatgtcca
ctgggttcgtgcccgaattgatcacaggcagcaacgctctgtcatcgttacaatcaacatgctaccctc
cgcgagatcatccgtgtttcaaacccggcagcttagttgccgttcttccgaatagcatcggtaacatga
gcaaagtctgccgccttacaacggctctcccgctgacgccgtcccggactgatgggctgcctgtatcga
gtggtgattttgtgccgagctgccggtcggggagctgttggctggctggtggcaggatatattgtggtg
taaacaaattgacgcttagacaacttaataacacattgcggacgtttttaatgtactgaattaacgccg
aattgaattcaggcctgtcgacgcccgggcggtaccgcgatcgctcgcgacctgcaggcataaagccgt
cagtgtccgcataaagaaccacccataatacccataatagctgtttgccatcgctaccttaggaccgtt
atagttaaccggtgaattcccgatctagtaacatagatgacaccgcgcgcgataatttatcctagtttg
cgcgctatattttgttttctatcgcgtattaaatgtataattgcgggactctaatcataaaaacccatc
tcataaataacgtcatgcattacatgttaattattacatgcttaacgtaattcaacagaaattatatga
taatcatcgcaagaccggcaacaggattcaatcttaagaaactttattgccaaatgtttgaacgatcgg
ccggccgagctcggtagcaattcccgaggctgtagccgacgatggtgccaccaggagagttgttgattc
attgtttgcctccctgctgcggtttttcaccgaagttcatgccagtccagcgtttttgcagcagaaaag
ccgccgacttcggtttgcggtcgcgagtgaagatccctttcttgttaccgccaacgcgcaatatgcctt
gcgaggtcgcaaaatcggcgaaattccatacctgttcaccgacgacggcgctgacgcgatcaaagacgc
ggtgatacatatccagccatgcacactgatactcttcactccacatgtcggtgtacattgagtgcagcc
cggctaacgtatccacgccgtattcggtgatgataatcggctgatgcagtttctcctgccaggccagaa
gttctttttccagtaccttctctgccgtttccaaatcgccgctttggacataccatccgtaataacggt
tcaggcacagcacatcaaagagatcgctgatggtatcggtgtgagcgtcgcagaacattacattgacgc
aggtgatcggacgcgtcgggtcgagtttacgcgttgcttccgccagtggcgcgaaatattcccgtgcac
cttgcggacgggtatccggttcgttggcaatactccacatcaccacgcttgggtggtttttgtcacgcg
ctatcagctctttaatcgcctgtaagtgcgcttgctgagtttccccgttgactgcctcttcgctgtaca
gttctttcggcttgttgcccgcttcgaaaccaatgcctaaagagaggttaaagccgacagcagcagttt
catcaatcaccacgatgccatgttcatctgcccagtcgagcatctcttcagcgtaagggtaatgcgagg
tacggtaggagttggccccaatccagtccattaatgcgtggtcgtgcaccatcagcacgttatcgaatc
ctttgccacgcaagtccgcatcttcatgacgaccaaagccagtaaagtagaacggtttgtggttaatca
ggaactgttcgcccttcactgccactgaccggatgccgacgcgaagcgggtagatatcacactctgtct
ggcttttggctgtgacgcacagttcatagagataaccttcacccggttgccagaggtgcggattcacca
cttgcaaagtcccgctagtgccttgtccagttgcaaccacctgttgatccgcatcacgcagttcaacgc
tgacatcaccattggccaccacctgccagtcaacagacgcgtggttacagtcttgcgcgacatgcgtca
ccacggtgatatcgtccacccaggtgttcggcgtggtgtagagcattacgctgcgatggattccggcat
agttaaagaaatcatggaagtaagactgctttttcttgccgttttcgtcggtaatcaccattcccggcg
ggatagtctgccagttcagttcgttgttcacacaaacggtgatacgtacacttttcccggcaataacat
acggcgtgacatcggcttcaaatggcgtatagccgccctgatgctccatcacttcctgattattgaccc
acactttgccgtaatgagtgaccgcatcgaaacgcagcacgatacgctggcctgcccaacctttcggta
taaagacttcgcgctgataccagacgttgcccgcataattacgaatatctgcatcggcgaactgatcgt
taaaactgcctggcacagcaattgcccggctttcttgtaacgcgctttcccaccaacgctgatcaattc
cacagttttcgcgatccagactgaatgcccacaggccgtcgagttttttgatttcacgggttggggttt
ctacaggacgtaacataagggactgacctacccggggatcctctagagccatggtgtttaaacgttaac
tgtaattgtaaatagtaattgtaatgttgtttgttgtttgttgttgttggtaattgttgtaaaaatact
cgaggtcctctccaaatgaaatgaacttccttatatagaggaagggtcttgcgaaggatagtgggattg
tgcgtcatcccttacgtcagtggagatatcacatcaatccacttgctttgaagacgtggttggaacgtc
ttcttttttccacgatgctcctcgtgggtgggggtccatctttgggaccactgtcggcagaggcatctt
caacgatggcctttcctttatcgcaatgatggcatttgtaggagccaccttccttttccactatcttca
caataaagtgacagatagctgggcaatggaatccgaggaggtttccggatattaccctttgttgaaaag
tctcaattgccctttggtcttctgagactgtatctttgatatttttggagtagacaagtgtgtcgtgct
ccaccatgttatcacatcaatccacttgctttgaagacgtggttggaacgtcttcttttttccacgatg
ctcctcgtgggtgggggtccatctttgggaccactgtcggcagaggcatcttcaacgatggcctttcct
ttatcgcaatgatggcatttgtaggagccaccttccttttccactatcttcacaataaagtgacagata
gctgggcaatggaatccgaggaggtttccggatattaccctttgttgaaaagtctcaattgccctttgg
tcttctgagactgtatctttgatatttttggagtagacaagtgtgtcgtgctccaccatgttcaagctt
gcggccgctcgctaccttaggaccgttatagttaattaccctgttatccctattaattaagagctcgct
accttaagagaggatatcggcgcgccgaattcgcgctctatcatagatgtcgctataaacctattcagc
acaatatattgttttcattttaatattgtacatataagtagtagggtacaatcagtaaattgaacggag
aatattattcataaaaatacgatagtaacgggtgatatattcattagaatgaaccgaaaccggcggtaa
ggatctgagctacacatgctcaggttttttacaacgtgcacaacagaattgaaagcaaatatcatgcga
tcataggcgtctcgcatatctcattaaagcagctggaagatttgatggatcctcatcagatctcggtga
cgggcaggaccggacggggcggtaccggcaggctgaagtccagctgccagaaacccacgtcatgccagt
tcccgtgcttgaagccggccgcccgcagcatgccgcggggggcatatccgagcgcctcgtgcatgcgca
cgctcgggtcgttgggcagcccgatgacagcgaccacgctcttgaagccctgtgcctccagggacttca
gcaggtgggtgtagagcgtggagcccagtcccgtccgctggtggcggggggagacgtacacggtcgact
cggccgtccagtcgtaggcgttgcgtgccttccaggggcccgcgtaggcgatgccggcgacctcgccgt
ccacctcggcgacgagccagggatagcgctcccgcagacggacgaggtcgtccgtccactcctgcggtt
cctgcggctcggtacggaagttgaccgtgcttgtctcgatgtagtggttgacgatggtgcagaccgccg
gcatgtccgcctcggtggcacggcggatgtcggccgggcgtcgttctgggctcatggtagatctgttta
aacgttaacggattgagagtgaatatgagactctaattggataccgaggggaatttatggaacgtcagt
ggagcatttttgacaagaaatatttgctagctgatagtgaccttaggcgacttttgaacgcgcaataat
ggtttctgacgtatgtgcttagctcattaaactccagaaacccgcggctgagtggctccttcaatcgtt
gcggttctgtcagttccaaacgtaaaacggcttgtcccgcgtcatcggcgggggtcataacgtgactcc
cttaattctccgctcatgatcaagcttggcgcgcctctagaatttaaatggatcctacgtactcgagaa
gcttagcttgagcttggatcagattgtcgtttcccgccttcagtttaaactatcagtgtttgacaggat
atattggcgggtaaacctaagagaaaagagcgtttattagaataacggatatttaaaagggcgtgaaaa
ggtttatccgttcgtccatttgtatgtgcatgccaaccacagggttcccctcgggatcaa

Sequence Annex 3: VpFAJ3163: 17062 bp.

atttgtatgtgcatgccaaccacagggttcccctcgggatcaaagtactttgatccaacccctccgctg
ctatagtgcagtcggcttctgacgttcagtgcagccgtcttctgaaaacgacatgtcgcacaagtccta
agttacgcgacaggctgccgccctgcccttttcctggcgttttcttgtcgcgtgttttagtcgcataaa
gtagaatacttgcgactagaaccggagacattacgccatgaacaagagcgccgccgctggcctgctggg
ctatgcccgcgtcagcaccgacgaccaggacttgaccaaccaacgggccgaactgcacgcggccggctg
caccaagctgttttccgagaagatcaccggcaccaggcgcgaccgcccggagctggccaggatgcttga
ccacctacgccctggcgacgttgtgacagtgaccaggctagaccgcctggcccgcagcacccgcgacct
actggacattgccgagcgcatccaggaggccggcgcgggcctgcgtagcctggcagagccgtgggccga
caccaccacgccggccggccgcatggtgttgaccgtgttcgccggcattgccgagttcgagcgttccct
aatcatcgaccgcacccggagcgggcgcgaggccgccaaggcccgaggcgtgaagtttggcccccgccc
taccctcaccccggcacagatcgcgcacgcccgcgagctgatcgaccaggaaggccgcaccgtgaaaga
ggcggctgcactgcttggcgtgcatcgctcgaccctgtaccgcgcacttgagcgcagcgaggaagtgac
gcccaccgaggccaggcggcgcggtgccttccgtgaggacgcattgaccgaggccgacgccctggcggc
cgccgagaatgaacgccaagaggaacaagcatgaaaccgcaccaggacggccaggacgaaccgtttttc
attaccgaagagatcgaggcggagatgatcgcggccgggtacgtgttcgagccgcccgcgcacgtctca
accgtgcggctgcatgaaatcctggccggtttgtctgatgccaagctggcggcctggccggccagcttg
gccgctgaagaaaccgagcgccgccgtctaaaaaggtgatgtgtatttgagtaaaacagcttgcgtcat
gcggtcgctgcgtatatgatgcgatgagtaaataaacaaatacgcaaggggaacgcatgaaggttatcg
ctgtacttaaccagaaaggcgggtcaggcaagacgaccatcgcaacccatctagcccgcgccctgcaac
tcgccggggccgatgttctgttagtcgattccgatccccagggcagtgcccgcgattgggcggccgtgc
gggaagatcaaccgctaaccgttgtcggcatcgaccgcccgacgattgaccgcgacgtgaaggccatcg
gccggcgcgacttcgtagtgatcgacggagcgccccaggcggcggacttggctgtgtccgcgatcaagg
cagccgacttcgtgctgattccggtgcagccaagcccttacgacatatgggccaccgccgacctggtgg
agctggttaagcagcgcattgaggtcacggatggaaggctacaagcggcctttgtcgtgtcgcgggcga
tcaaaggcacgcgcatcggcggtgaggttgccgaggcgctggccgggtacgagctgcccattcttgagt
cccgtatcacgcagcgcgtgagctacccaggcactgccgccgccggcacaaccgttcttgaatcagaac
ccgagggcgacgctgcccgcgaggtccaggcgctggccgctgaaattaaatcaaaactcatttgagtta
atgaggtaaagagaaaatgagcaaaagcacaaacacgctaagtgccggccgtccgagcgcacgcagcag
caaggctgcaacgttggccagcctggcagacacgccagccatgaagcgggtcaactttcagttgccggc
ggaggatcacaccaagctgaagatgtacgcggtacgccaaggcaagaccattaccgagctgctatctga
atacatcgcgcagctaccagagtaaatgagcaaatgaataaatgagtagatgaattttagcggctaaag
gaggcggcatggaaaatcaagaacaaccaggcaccgacgccgtggaatgccccatgtgtggaggaacgg
gcggttggccaggcgtaagcggctgggttgtctgccggccctgcaatggcactggaacccccaagcccg
aggaatcggcgtgacggtcgcaaaccatccggcccggtacaaatcggcgcggcgctgggtgatgacctg
gtggagaagttgaaggccgcgcaggccgcccagcggcaacgcatcgaggcagaagcacgccccggtgaa
tcgtggcaagcggccgctgatcgaatccgcaaagaatcccggcaaccgccggcagccggtgcgccgtcg
attaggaagccgcccaagggcgacgagcaaccagattttttcgttccgatgctctatgacgtgggcacc
cgcgatagtcgcagcatcatggacgtggccgttttccgtctgtcgaagcgtgaccgacgagctggcgag
gtgatccgctacgagcttccagacgggcacgtagaggtttccgcagggccggccggcatggccagtgtg
tgggattacgacctggtactgatggcggtttcccatctaaccgaatccatgaaccgataccgggaaggg
aagggagacaagcccggccgcgtgttccgtccacacgttgcggacgtactcaagttctgccggcgagcc
gatggcggaaagcagaaagacgacctggtagaaacctgcattcggttaaacaccacgcacgttgccatg
cagcgtacgaagaaggccaagaacggccgcctggtgacggtatccgagggtgaagccttgattagccgc
tacaagatcgtaaagagcgaaaccgggcggccggagtacatcgagatcgagctagctgattggatgtac
cgcgagatcacagaaggcaagaacccggacgtgctgacggttcaccccgattactttttgatcgatccc
ggcatcggccgttttctctaccgcctggcacgccgcgccgcaggcaaggcagaagccagatggttgttc
aagacgatctacgaacgcagtggcagcgccggagagttcaagaagttctgtttcaccgtgcgcaagctg
atcgggtcaaatgacctgccggagtacgatttgaaggaggaggcggggcaggctggcccgatcctagtc
atgcgctaccgcaacctgatcgagggcgaagcatccgccggttcctaatgtacggagcagatgctaggg
caaattgccctagcaggggaaaaaggtcgaaaaggtctctttcctgtggatagcacgtacattgggaac
ccaaagccgtacattgggaaccggaacccgtacattgggaacccaaagccgtacattgggaaccggtca
cacatgtaagtgactgatataaaagagaaaaaaggcgatttttccgcctaaaactctttaaaacttatt
aaaactcttaaaacccgcctggcctgtgcataactgtctggccagcgcacagccgaagagctgcaaaaa
gcgcctacccttcggtcgctgcgctccctacgccccgccgcttcgcgtcggcctatcgcggccgctggc
cgctcaaaaatggctggcctacggccaggcaatctaccagggcgcggacaagccgcgccgtcgccactc
gaccgccggcgcccacatcaaggcaccctgcctcgcgcgtttcggtgatgacggtgaaaacctctgaca
catgcagctcccggagacggtcacagcttgtctgtaagcggatgccgggagcagacaagcccgtcaggg
cgcgtcagcgggtgttggcgggtgtcggggcgcagccatgacccagtcacgtagcgatagcggagtgta
tactggcttaactatgcggcatcagagcagattgtactgagagtgcaccatatgcggtgtgaaataccg
cacagatgcgtaaggagaaaataccgcatcaggcgctcttccgcttcctcgctcactgactcgctgcgc
tcggtcgttcggctgcggcgagcggtatcagctcactcaaaggcggtaatacggttatccacagaatca
ggggataacgcaggaaagaacatgtgagcaaaaggccagcaaaaggccaggaaccgtaaaaaggccgcg
ttgctggcgtttttccataggctccgcccccctgacgagcatcacaaaaatcgacgctcaagtcagagg
tggcgaaacccgacaggactataaagataccaggcgtttccccctggaagctccctcgtgcgctctcct
gttccgaccctgccgcttaccggatacctgtccgcctttctcccttcgggaagcgtggcgctttctcat
agctcacgctgtaggtatctcagttcggtgtaggtcgttcgctccaagctgggctgtgtgcacgaaccc
cccgttcagcccgaccgctgcgccttatccggtaactatcgtcttgagtccaacccggtaagacacgac
ttatcgccactggcagcagccactggtaacaggattagcagagcgaggtatgtaggcggtgctacagag
ttcttgaagtggtggcctaactacggctacactagaaggacagtatttggtatctgcgctctgctgaag
ccagttaccttcggaaaaagagttggtagctcttgatccggcaaacaaaccaccgctggtagcggtggt
ttttttgtttgcaagcagcagattacgcgcagaaaaaaaggatctcaagaagatcctttgatcttttct
acggggtctgacgctcagtggaacgaaaactcacgttaagggattttggtcatgcatgatatatctccc
aatttgtgtagggcttattatgcacgcttaaaaataataaaagcagacttgacctgatagtttggctgt
gagcaattatgtgcttagtgcatctaatcgcttgagttaacgccggcgaagcggcgtcggcttgaacga
atttctagctagacattatttgccgactaccttggtgatctcgcctttcacgtagtggacaaattcttc
caactgatctgcgcgcgaggccaagcgatcttcttcttgtccaagataagcctgtctagcttcaagtat
gacgggctgatactgggccggcaggcgctccattgcccagtcggcagcgacatccttcggcgcgatttt
gccggttactgcgctgtaccaaatgcgggacaacgtaagcactacatttcgctcatcgccagcccagtc
gggcggcgagttccatagcgttaaggtttcatttagcgcctcaaatagatcctgttcaggaaccggatc
aaagagttcctccgccgctggacctaccaaggcaacgctatgttctcttgcttttgtcagcaagatagc
cagatcaatgtcgatcgtggctggctcgaagatacctgcaagaatgtcattgcgctgccattctccaaa
ttgcagttcgcgcttagctggataacgccacggaatgatgtcgtcgtgcacaacaatggtgacttctac
agcgcggagaatctcgctctctccaggggaagccgaagtttccaaaaggtcgttgatcaaagctcgccg
cgttgtttcatcaagccttacggtcaccgtaaccagcaaatcaatatcactgtgtggcttcaggccgcc
atccactgcggagccgtacaaatgtacggccagcaacgtcggttcgagatggcgctcgatgacgccaac
tacctctgatagttgagtcgatacttcggcgatcaccgcttcccccatgatgtttaactttgttttagg
gcgactgccctgctgcgtaacatcgttgctgctccataacatcaaacatcgacccacggcgtaacgcgc
ttgctgcttggatgcccgaggcatagactgtaccccaaaaaaacatgtcataacaagaagccatgaaaa
ccgccactgcgccgttaccaccgctgcgttcggtcaaggttctggaccagttgcgtgacggcagttacg
ctacttgcattacagcttacgaaccgaacgaggcttatgtccactgggttcgtgcccgaattgatcaca
ggcagcaacgctctgtcatcgttacaatcaacatgctaccctccgcgagatcatccgtgtttcaaaccc
ggcagcttagttgccgttcttccgaatagcatcggtaacatgagcaaagtctgccgccttacaacggct
ctcccgctgacgccgtcccggactgatgggctgcctgtatcgagtggtgattttgtgccgagctgccgg
tcggggagctgttggctggctggtggcaggatatattgtggtgtaaacaaattgacgcttagacaactt
aataacacattgcggacgtttttaatgtactgaattaacgccgaattgaattcaggcctgtcgactcta
gaaaaccaatatatttccaaatgaaaaaaaaatctgataaaaagttgactttaaaaaaggtatcaataa
atgtatgcatttctcactagccttaaactctgcatgaagtgtttgatgagcagatgaagacaacatcat
ttctagtttcagaaataataacagcatcaaaaccgcagctgtaactccactgagctcacgttaagtttt
gatgtgtgaatatctgacagaactgacataatgagcactgcaaggatatcagacaagtcaaaatgaaga
cagacaaaagtattttttaatataaaaatggtctttatttcttcaatacaaggtaaactactattgcag
tttaagaccaacacaaaagttggacagcaaattgcttaacagtctcctaaaggctgaaaaaaaggaacc
catgaaagctaaaagttatgcagtatttcaagtataacatctaaaaatgatgaaacgatccctaaaggt
agagattaactaagtacttctgctgaaaatgtattaaaatccgcagttgctaggataccatcttacctt
gttgagaaatacaggtctccggcaacgcaacattcagcagactctttggcctgctggaatcaggaaact
gcttactatatacacatataaatcctttggagttgggcattctgagagacatccatttcctgacatttt
gcagtgcaactctgcattccaactcagacaagctcccatgctgtatttcaaagccatttcttgaatagt
ttacccagacatccttgtgcaaattgggaatgaggaaatgcaatggtacaggaagacaatacagcctta
tgtttagaaagtcagcagcgctggtaatcttcataaaaatgtaactgttttccaaataggaatgtattt
cacttgtaaaacacctggtcctttttatattacttttttttttttttaaggacacctgcactaatttgc
aatcacttgtatttataaaagcacacgcactcctcattttcttacatttgaagatcagcagaatgtctc
tttcataatgtaataatcatatgcacagtttaaaatattttctattacaaaatacagtacacaagaggg
tgaggccaaagtctattacttgaatatattccaaagtgtcagcactgggggtgtaaaattacattacat
ggtatgaataggcggaattcttttacaactgaaatgctcgatttcattgggatcaaaggtaagtactgt
ttactatcttcaagagacttcaatcaagtcggtgtatttccaaagaagcttaaaagattgaagcacaga
cacaggccacaccagagcctacacctgctgcaataagtggtgctatagaaaggattcaggaactaacaa
gtgcataatttacaaatagagatgctttatcatactttgcccaacatgggaaaaaagacatcccatgag
aatatccaactgaggaacttctctgtttcatagtaactcatctactactgctaagatggtttgaaaagt
acccagcaggtgagatatgttcgggaggtggctgtgtggcagcgtgtcccaacacgacacaaagcaccc
cacccctatctgcaatgctcactgcaaggcagtgccgtaaacagctgcaacaggcatcacttctgcata
aatgctgtgactcgttagcatgctgcaactgtgtttaaaacctatgcactccgttaccaaaataattta
agtcccaaataaatccatgcagcttgcttcctatgccaacatattttagaaagtattcattcttcttta
agaatatgcacgtggatctacacttcctgggatctgaagcgatttatacctcagttgcagaagcagttt
agtgtcctggatctgggaaggcagcagcaaacgtgcccgttttacatttgaacccatgtgacaacccgc
cttactgagcatcgctctaggaaatttaaggctgtatccttacaacacaagaaccaacgacagactgca
tataaaattctataaataaaaataggagtgaagtctgtttgacctgtacacacagagcatagagataaa
aaaaaaaggaaatcaggaattacgtatttctataaatgccatatatttttactagaaacacagatgaca
agtatatacaacatgtaaatccgaagttatcaacatgttaactaggaaaacatttacaagcatttgggt
atgcaactagatcatcaggtaaaaaatcccattagaaaaatctaagcctcgccagtttcaaaggaaaaa
aaccagagaacgctcactacttcaaaggaaaaaaaataaagcatcaagctggcctaaacttaataaggt
atctcatgtaacaacagctatccaagctttcaagccacactataaataaaaacctcaagttccgatcaa
cgttttccataatgcaatcagaaccaaaggcattggcacagaaagcaaaaagggaatgaaagaaaaggg
ctgtacagtttccaaaaggttcttcttttgaagaaatgtttctgacctgtcaaaacatacagtccagta
gaaattttactaagaaaaaagaacaccttacttaaaaaaaaaaaacaacaaaaaaaacaggcaaaaaaa
cctctcctgtcactgagctgccaccacccaaccaccacctgctgtgggctttgtctcccaagacaaagg
acacacagccttatccaatattcaacattacttataaaaacgctgatcagaagaaataccaagtatttc
ctcagagactgttatatcctttcatcggcaacaagagatgaaatacaacagagtgaatatcaaagaagg
cggcaggagccaccgtggcaccatcaccgggcagtgcagtgcccaactgccgttttctgagcacgcata
ggaagccgtcagtcacatgtaataaaccaaaacctggtacagttatattatggatccccgggtaccgcg
atcgctcgcgacctgcaggcataaagccgtcagtgtccgcataaagaaccacccataatacccataata
gctgtttgccatcgctaccttaggaccgttatagttaaccggtgaattcccgatctagtaacatagatg
acaccgcgcgcgataatttatcctagtttgcgcgctatattttgttttctatcgcgtattaaatgtata
attgcgggactctaatcataaaaacccatctcataaataacgtcatgcattacatgttaattattacat
gcttaacgtaattcaacagaaattatatgataatcatcgcaagaccggcaacaggattcaatcttaaga
aactttattgccaaatgtttgaacgatcggccggccgagctcggtagcaattcccgaggctgtagccga
cgatggtgccaccaggagagttgttgattcattgtttgcctccctgctgcggtttttcaccgaagttca
tgccagtccagcgtttttgcagcagaaaagccgccgacttcggtttgcggtcgcgagtgaagatccctt
tcttgttaccgccaacgcgcaatatgccttgcgaggtcgcaaaatcggcgaaattccatacctgttcac
cgacgacggcgctgacgcgatcaaagacgcggtgatacatatccagccatgcacactgatactcttcac
tccacatgtcggtgtacattgagtgcagcccggctaacgtatccacgccgtattcggtgatgataatcg
gctgatgcagtttctcctgccaggccagaagttctttttccagtaccttctctgccgtttccaaatcgc
cgctttggacataccatccgtaataacggttcaggcacagcacatcaaagagatcgctgatggtatcgg
tgtgagcgtcgcagaacattacattgacgcaggtgatcggacgcgtcgggtcgagtttacgcgttgctt
ccgccagtggcgcgaaatattcccgtgcaccttgcggacgggtatccggttcgttggcaatactccaca
tcaccacgcttgggtggtttttgtcacgcgctatcagctctttaatcgcctgtaagtgcgcttgctgag
tttccccgttgactgcctcttcgctgtacagttctttcggcttgttgcccgcttcgaaaccaatgccta
aagagaggttaaagccgacagcagcagtttcatcaatcaccacgatgccatgttcatctgcccagtcga
gcatctcttcagcgtaagggtaatgcgaggtacggtaggagttggccccaatccagtccattaatgcgt
ggtcgtgcaccatcagcacgttatcgaatcctttgccacgcaagtccgcatcttcatgacgaccaaagc
cagtaaagtagaacggtttgtggttaatcaggaactgttcgcccttcactgccactgaccggatgccga
cgcgaagcgggtagatatcacactctgtctggcttttggctgtgacgcacagttcatagagataacctt
cacccggttgccagaggtgcggattcaccacttgcaaagtcccgctagtgccttgtccagttgcaacca
cctgttgatccgcatcacgcagttcaacgctgacatcaccattggccaccacctgccagtcaacagacg
cgtggttacagtcttgcgcgacatgcgtcaccacggtgatatcgtccacccaggtgttcggcgtggtgt
agagcattacgctgcgatggattccggcatagttaaagaaatcatggaagtaagactgctttttcttgc
cgttttcgtcggtaatcaccattcccggcgggatagtctgccagttcagttcgttgttcacacaaacgg
tgatacgtacacttttcccggcaataacatacggcgtgacatcggcttcaaatggcgtatagccgccct
gatgctccatcacttcctgattattgacccacactttgccgtaatgagtgaccgcatcgaaacgcagca
cgatacgctggcctgcccaacctttcggtataaagacttcgcgctgataccagacgttgcccgcataat
tacgaatatctgcatcggcgaactgatcgttaaaactgcctggcacagcaattgcccggctttcttgta
acgcgctttcccaccaacgctgatcaattccacagttttcgcgatccagactgaatgcccacaggccgt
cgagttttttgatttcacgggttggggtttctacaggacgtaacataagggactgacctacccggggat
cctctagagccatggtgtttaaacgttaactgtaattgtaaatagtaattgtaatgttgtttgttgttt
gttgttgttggtaattgttgtaaaaatactcgaggtcctctccaaatgaaatgaacttccttatataga
ggaagggtcttgcgaaggatagtgggattgtgcgtcatcccttacgtcagtggagatatcacatcaatc
cacttgctttgaagacgtggttggaacgtcttcttttttccacgatgctcctcgtgggtgggggtccat
ctttgggaccactgtcggcagaggcatcttcaacgatggcctttcctttatcgcaatgatggcatttgt
aggagccaccttccttttccactatcttcacaataaagtgacagatagctgggcaatggaatccgagga
ggtttccggatattaccctttgttgaaaagtctcaattgccctttggtcttctgagactgtatctttga
tatttttggagtagacaagtgtgtcgtgctccaccatgttatcacatcaatccacttgctttgaagacg
tggttggaacgtcttcttttttccacgatgctcctcgtgggtgggggtccatctttgggaccactgtcg
gcagaggcatcttcaacgatggcctttcctttatcgcaatgatggcatttgtaggagccaccttccttt
tccactatcttcacaataaagtgacagatagctgggcaatggaatccgaggaggtttccggatattacc
ctttgttgaaaagtctcaattgccctttggtcttctgagactgtatctttgatatttttggagtagaca
agtgtgtcgtgctccaccatgttcaagcttgcggccgctcgctaccttaggaccgttatagttaattac
cctgttatccctattaattaagagctcgctaccttaagagagaccggtgaattcgcgctctatcataga
tgtcgctataaacctattcagcacaatatattgttttcattttaatattgtacatataagtagtagggt
acaatcagtaaattgaacggagaatattattcataaaaatacgatagtaacgggtgatatattcattag
aatgaaccgaaaccggcggtaaggatctgagctacacatgctcaggttttttacaacgtgcacaacaga
attgaaagcaaatatcatgcgatcataggcgtctcgcatatctcattaaagcagctggaagatttgatg
gatcctcatcagatctcggtgacgggcaggaccggacggggcggtaccggcaggctgaagtccagctgc
cagaaacccacgtcatgccagttcccgtgcttgaagccggccgcccgcagcatgccgcggggggcatat
ccgagcgcctcgtgcatgcgcacgctcgggtcgttgggcagcccgatgacagcgaccacgctcttgaag
ccctgtgcctccagggacttcagcaggtgggtgtagagcgtggagcccagtcccgtccgctggtggcgg
ggggagacgtacacggtcgactcggccgtccagtcgtaggcgttgcgtgccttccaggggcccgcgtag
gcgatgccggcgacctcgccgtccacctcggcgacgagccagggatagcgctcccgcagacggacgagg
tcgtccgtccactcctgcggttcctgcggctcggtacggaagttgaccgtgcttgtctcgatgtagtgg
ttgacgatggtgcagaccgccggcatgtccgcctcggtggcacggcggatgtcggccgggcgtcgttct
gggctcatggtagatctgtttaaacgttaacggattgagagtgaatatgagactctaattggataccga
ggggaatttatggaacgtcagtggagcatttttgacaagaaatatttgctagctgatagtgaccttagg
cgacttttgaacgcgcaataatggtttctgacgtatgtgcttagctcattaaactccagaaacccgcgg
ctgagtggctccttcaatcgttgcggttctgtcagttccaaacgtaaaacggcttgtcccgcgtcatcg
gcgggggtcataacgtgactcccttaattctccgctcatgatcaagcttgcggccgcggcgcgcctcta
gaaaaccaatatatttccaaatgaaaaaaaaatctgataaaaagttgactttaaaaaaggtatcaataa
atgtatgcatttctcactagccttaaactctgcatgaagtgtttgatgagcagatgaagacaacatcat
ttctagtttcagaaataataacagcatcaaaaccgcagctgtaactccactgagctcacgttaagtttt
gatgtgtgaatatctgacagaactgacataatgagcactgcaaggatatcagacaagtcaaaatgaaga
cagacaaaagtattttttaatataaaaatggtctttatttcttcaatacaaggtaaactactattgcag
tttaagaccaacacaaaagttggacagcaaattgcttaacagtctcctaaaggctgaaaaaaaggaacc
catgaaagctaaaagttatgcagtatttcaagtataacatctaaaaatgatgaaacgatccctaaaggt
agagattaactaagtacttctgctgaaaatgtattaaaatccgcagttgctaggataccatcttacctt
gttgagaaatacaggtctccggcaacgcaacattcagcagactctttggcctgctggaatcaggaaact
gcttactatatacacatataaatcctttggagttgggcattctgagagacatccatttcctgacatttt
gcagtgcaactctgcattccaactcagacaagctcccatgctgtatttcaaagccatttcttgaatagt
ttacccagacatccttgtgcaaattgggaatgaggaaatgcaatggtacaggaagacaatacagcctta
tgtttagaaagtcagcagcgctggtaatcttcataaaaatgtaactgttttccaaataggaatgtattt
cacttgtaaaacacctggtcctttttatattacttttttttttttttaaggacacctgcactaatttgc
aatcacttgtatttataaaagcacacgcactcctcattttcttacatttgaagatcagcagaatgtctc
tttcataatgtaataatcatatgcacagtttaaaatattttctattacaaaatacagtacacaagaggg
tgaggccaaagtctattacttgaatatattccaaagtgtcagcactgggggtgtaaaattacattacat
ggtatgaataggcggaattcttttacaactgaaatgctcgatttcattgggatcaaaggtaagtactgt
ttactatcttcaagagacttcaatcaagtcggtgtatttccaaagaagcttaaaagattgaagcacaga
cacaggccacaccagagcctacacctgctgcaataagtggtgctatagaaaggattcaggaactaacaa
gtgcataatttacaaatagagatgctttatcatactttgcccaacatgggaaaaaagacatcccatgag
aatatccaactgaggaacttctctgtttcatagtaactcatctactactgctaagatggtttgaaaagt
acccagcaggtgagatatgttcgggaggtggctgtgtggcagcgtgtcccaacacgacacaaagcaccc
cacccctatctgcaatgctcactgcaaggcagtgccgtaaacagctgcaacaggcatcacttctgcata
aatgctgtgactcgttagcatgctgcaactgtgtttaaaacctatgcactccgttaccaaaataattta
agtcccaaataaatccatgcagcttgcttcctatgccaacatattttagaaagtattcattcttcttta
agaatatgcacgtggatctacacttcctgggatctgaagcgatttatacctcagttgcagaagcagttt
agtgtcctggatctgggaaggcagcagcaaacgtgcccgttttacatttgaacccatgtgacaacccgc
cttactgagcatcgctctaggaaatttaaggctgtatccttacaacacaagaaccaacgacagactgca
tataaaattctataaataaaaataggagtgaagtctgtttgacctgtacacacagagcatagagataaa
aaaaaaaggaaatcaggaattacgtatttctataaatgccatatatttttactagaaacacagatgaca
agtatatacaacatgtaaatccgaagttatcaacatgttaactaggaaaacatttacaagcatttgggt
atgcaactagatcatcaggtaaaaaatcccattagaaaaatctaagcctcgccagtttcaaaggaaaaa
aaccagagaacgctcactacttcaaaggaaaaaaaataaagcatcaagctggcctaaacttaataaggt
atctcatgtaacaacagctatccaagctttcaagccacactataaataaaaacctcaagttccgatcaa
cgttttccataatgcaatcagaaccaaaggcattggcacagaaagcaaaaagggaatgaaagaaaaggg
ctgtacagtttccaaaaggttcttcttttgaagaaatgtttctgacctgtcaaaacatacagtccagta
gaaattttactaagaaaaaagaacaccttacttaaaaaaaaaaaacaacaaaaaaaacaggcaaaaaaa
cctctcctgtcactgagctgccaccacccaaccaccacctgctgtgggctttgtctcccaagacaaagg
acacacagccttatccaatattcaacattacttataaaaacgctgatcagaagaaataccaagtatttc
ctcagagactgttatatcctttcatcggcaacaagagatgaaatacaacagagtgaatatcaaagaagg
cggcaggagccaccgtggcaccatcaccgggcagtgcagtgcccaactgccgttttctgagcacgcata
ggaagccgtcagtcacatgtaataaaccaaaacctggtacagttatattatggatcctacgtactcgag
aagcttagcttgagcttggatcagattgtcgtttcccgccttcagtttaaactatcagtgtttgacagg
atatattggcgggtaaacctaagagaaaagagcgtttattagaataacggatatttaaaagggcgtgaa
aaggtttatccgttcgtcc

1-31. (canceled)

32. A method of achieving enhanced expression of a target nucleotide sequence in a transgenic organism, which method comprises the steps of:
(i) providing an organism in which post-transcriptional gene silencing (PTGS) is suppressed,
(ii) associating said target nucleotide sequence with one or more heterologous Matrix Attachment Region (MARs), and
(iii) causing or permitting expression from the target nucleotide sequence in the organism.

33. A method as claimed in claim 32 wherein in (ii) two MARs are associated with the target nucleotide sequence in positions flanking it.

34. A method as claimed in claim 32 wherein the target nucleotide sequence is operably linked to a heterologous promoter or enhancer sequence.

35. A method as claimed in claim 34 wherein (ii) comprises the step of operably linking said target nucleotide sequence with a heterologous promoter or enhancer sequence.

36. A method as claimed in claim 32 wherein in (ii) the or each of the MARs is introduced to and associated with a target nucleotide sequence which is within a pre-existing gene present in the genome of the organism.

37. A method as claimed in claim 36 wherein the or each MAR is less than 500, 200, 150, 100, or 50 nucleotides upstream of a promoter or downstream of a terminator of the gene.

38. A method as claimed in claim 36 wherein (ii) comprises the steps of:
(iia) providing a target nucleic acid construct comprising (a) a promoter, and (b) one or more Matrix Attachment Regions (MARs) associated therewith,
(iib)introducing said target construct into a cell of the organism, such that the promoter becomes operably linked to a target nucleotide sequence which is within a pre-existing gene present in the genome of the organism.

39. A method as claimed claim 32 wherein the target nucleotide sequence is endogenous to the organism.

40. A method as claimed in claim 32 wherein (ii) comprises the steps of:
(iia) providing a target nucleic acid construct comprising (a) an expression cassette including the target nucleotide sequence operably linked to a promoter, and (b) one or more Matrix Attachment Regions (MARs) associated therewith,
(iib) introducing said target construct into a cell of the organism,

41. A method as claimed in claim 40 wherein 1 MAR is associated with the expression cassette 5′ of the cassette.

42. A method as claimed in claim 41 wherein the or each MAR is less than 500, 200, 150, 100, or 50 nucleotides upstream of a promoter or downstream of a terminator of the expression cassette.

43. A method as claimed in claim 40 wherein 2 MARs are associated with the expression cassette which flank the target nucleotide sequence.

44. A method as claimed in claim 43 wherein the or each MAR is less than 500, 200, 150, 100, or 50 nucleotides upstream of a promoter or downstream of a terminator of the expression cassette.

45. A method as claimed in claim 38 wherein the target construct is a vector which comprises border sequences which permit the transfer and integration of the MARs into the organism genome.

46. A method as claimed in claim 45 wherein the target construct is a plant binary vector.

47. A method of transforming a plant cell involving introduction of a construct as claimed in claim 45 such as to cause recombination between the vector and the plant cell genome.

48. A method as claimed in claim 47 which comprises the step of regenerating a plant from the transformed plant cell.

49. A method as claimed in claim 40 wherein the target construct is a vector which comprises border sequences which permit the transfer and integration of the MARs into the organism genome.

50. A method as claimed in claim 49 wherein the target construct is a plant binary vector.

51. A method of transforming a plant cell involving introduction of a construct as claimed in claim 49 such as to cause recombination between the vector and the plant cell genome.

52. A method as claimed in claim 51 which comprises the step of regenerating a plant from the transformed plant cell.

53. A method as claimed in claim 32 wherein (i) comprises the step of suppressing PTGS in the organism.

54. A method as claimed in claim 53 wherein step (ii) precedes step (i).

55. A method as claimed in claim 32 wherein the organism in which PTGS is suppressed is one which is deficient in one or more genes required to support PTGS.

56. A method as claimed in claim 55 wherein the organism is a plant and the genes required to support PTGS are selected from: SGS2; SDE1; SGS3; SDE3; AGO1; WEX.

57. A method as claimed in claim 32 wherein one or more genes required to support PTGS are subject to PTGS.

58. A method as claimed in claim 57 wherein the organism is a plant and the genes required to support PTGS are selected from: SGS2; SDE1; SGS3; SDE3; AGO1; WEX.

59. A method as claimed in claim 32 wherein PTGS is suppressed by one or more viral suppressors of gene silencing.

60. A transgenic non-human organism obtained or obtainable by a method as claimed in claim 32.

61. A transgenic organism as claimed in claim 60 in which a heterologous target nucleotide sequence is expressed at an enhanced level, wherein the organism is deficient in one or more genes required to support PTGS, which organism includes in its genome (a) an expression cassette including the target nucleotide sequence operably linked to a promoter, and (b) one or more heterologous Matrix Attachment Regions (MARs) associated therewith.

62. A method as claimed in claim 32 wherein expression is enhanced at least 5, 10, 15, 20, 25, or 30-fold.

63. A method for generating a target protein, which method comprises the steps of performing a method as claimed in claim 32 wherein the organism is a plant, and harvesting a tissue in which the target protein has been expressed and isolating the target protein from the tissue.

64. A method of producing a transgenic organism in which a target nucleotide sequence is expressed at an enhanced level, which method comprises the steps of:
(i) providing an organism in which post-transcriptional gene silencing (PTGS) is suppressed,
(ii) associating said target nucleotide sequence with one or more heterologous Matrix Attachment Region (MARs), and optionally:
(iii) causing or permitting expression from the target nucleotide sequence in the organism.

65. A target nucleic acid construct for achieving enhanced levels of expression of said target nucleic acid comprising (a) an expression cassette including the target nucleotide sequence operably linked to a promoter, and (b) one or more Matrix Attachment Regions (MARs) associated therewith, when used in connection with a cell or organism undergoing suppression of PTGS.

66. The construct according to claim 65 wherein 2 MARs are associated with the expression cassette which flank the target nucleotide sequence.

67. The construct according to claim 65 wherein the target construct is a vector which comprises border sequences which permit the transfer and integration of the MARs into the organism genome.

68. A composition for use in a cell or organism which comprises a target nucleic acid construct for achieving enhanced levels of expression of said target nucleic acid comprising (a) an expression cassette including the target nucleotide sequence operably linked to a promoter, and (b) one or more Matrix Attachment Regions (MARs) associated therewith, when used in connection with a cell or organism undergoing suppression of PTGS.
